SEQUENCE LISTING (24 sequences)

SEQ ID NO: 1 - DNA, 21 bp, Artificial
  An artificially synthesized primer sequence
ccatcatgaagtgtgacgtgg

SEQ ID NO: 2 - DNA, 21 bp, Artificial
  An artificially synthesized primer sequence
gtccgcctagaagcatttgcg

SEQ ID NO: 3 - DNA, 21 bp, Artificial
  An artificially synthesized primer sequence
ccatcatgaagtgtgacgtgg

SEQ ID NO: 4 - DNA, 21 bp, Artificial
  An artificially synthesized primer sequence
gtccgcctagaagcatttgcg

SEQ ID NO: 5 - DNA, 24 bp, Artificial
  An artificially synthesized primer sequence
gagactcagaacacaacctacctg

SEQ ID NO: 6 - DNA, 24 bp, Artificial
  An artificially synthesized primer sequence
agccagtactccaatcatgatgct

SEQ ID NO: 7 - DNA, 24 bp, Artificial
  An artificially synthesized primer sequence
caggatccactgcaaggaacaaca

SEQ ID NO: 8 - DNA, 24 bp, Artificial
  An artificially synthesized primer sequence
catgtatctttcagttacattctc

SEQ ID NO: 9 - DNA, 21 bp, Artificial
  An artificially synthesized primer sequence
ccatcatgaagtgtgacgtgg

SEQ ID NO: 10 - DNA, 21 bp, Artificial
  An artificially synthesized primer sequence
gtccgcctagaagcatttgcg

SEQ ID NO: 11 - DNA, 20 bp, Artificial
  An artificially synthesized primer sequence
agagatcagcgcctgagaag

SEQ ID NO: 12 - DNA, 20 bp, Artificial
  An artificially synthesized primer sequence
tgggctctaaattggctcac

SEQ ID NO: 13 - DNA, 20 bp, Artificial
  An artificially synthesized primer sequence
cacagccccaggtacaaact

SEQ ID NO: 14 - DNA, 20 bp, Artificial
  An artificially synthesized primer sequence
tttgctttgtcagcatctgg

SEQ ID NO: 15 - DNA, 20 bp, Artificial
  An artificially synthesized primer sequence
ctgccaaactgcaacaagaa

SEQ ID NO: 16 - DNA, 20 bp, Artificial
  An artificially synthesized primer sequence
tatctggcagttggggtctc

SEQ ID NO: 17 - DNA, 19 bp, Artificial
  An artificially synthesized primer sequence
aagcctgcacgaatgagtt

SEQ ID NO: 18 - DNA, 20 bp, Artificial
  An artificially synthesized primer sequence
ggagggatgaggatcacaga

SEQ ID NO: 19 - DNA, 20 bp, Artificial
  An artificially synthesized primer sequence
aggcatacgccaagatcaac

SEQ ID NO: 20 - DNA, 20 bp, Artificial
  An artificially synthesized primer sequence
gccaaggagagagccttttt

SEQ ID NO: 21 - DNA, 24 bp, Artificial
  An artificially synthesized primer sequence
caggatccactgcaaggaacaaca

SEQ ID NO: 22 - DNA, 24 bp, Artificial
  An artificially synthesized primer sequence
catgtatctttcagttacattctc

SEQ ID NO: 23 - DNA, 21 bp, Artificial
  An artificially synthesized primer sequence
ccatcatgaagtgtgacgtgg

SEQ ID NO: 24 - DNA, 21 bp, Artificial
  An artificially synthesized primer sequence
gtccgcctagaagcatttgcg

(19) United States
(12) Patent Application Publication
(10) Pub. No.: US 2006/0094007 A1
(43) Pub. Date: May 4, 2006

(54) Method of identifying pancreatic ductal carcinoma-specific gene using pancreatic ductal cells, method of testing for pdc using said genes, and method of screening pharmaceutical candidate compounds for treating or preventing pdc

(75) Inventor: Hiroyuki Mano, Tochigi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD, Osaka (JP)

(21) Appl. No.: 10/514,235

(22) PCT Filed: May 22, 2003
(86) PCT No.: PCT/JP03/06398

§ 371 Date: Sep. 6, 2005

Related U.S. Application Data
(60) Provisional application No. 60/382,022, filed on May 22, 2002.

(51) Int. Cl. C12Q 1/68

(52) U.S. Cl. 435/6

(57) ABSTRACT

A method for identifying a pancreatic ductal carcinoma-specific gene made up of the steps of: a) preparing pancreatic ductal cells from a pancreatic ductal carcinoma patient and a normal individual; b) detecting the gene expression in the pancreatic ductal cells prepared from the pancreatic ductal carcinoma patient and the gene expression in the pancreatic ductal cells prepared from the normal individual; c) comparing the gene expression in the pancreatic ductal cells prepared from the pancreatic ductal carcinoma patient with the gene expression in the pancreatic ductal cells prepared from the normal individual; and d) identifying a gene specifically expressed in the pancreatic ductal carcinoma patient and a gene specifically expressed in the normal individual.

### TECHNICAL FIELD

The present invention relates to a method of identifying a pancreatic ductal carcinoma-specific gene using pancreatic ductal cells, and a method of testing for pancreatic ductal carcinoma using the pancreatic ductal carcinoma-specific gene that is identified by the method, and a method of screening a pharmaceutical candidate compound for treating or preventing pancreatic ductal carcinoma.

### BACKGROUND ART

Pancreatic carcinoma remains the most intractable disorder among the gastoenterological malignancies with a five-year survival rate <5% (Bornman, P. C. and Beckingham, I. J. Pancreatic tumours. Brit. Med. J., 322: 721-723, 2001.; Rosewicz, S. and Wiedenmann, B. Pancreatic carcinoma. Lancet, 349: 485-489, 1997.). More than 90% of pancreas carcinoma found in patients is adenocarcinoma of ductal cell-origin. Partly due to the lack of disease-specific symptoms, it is rare to find patients at an early stage of pancreatic carcinoma, and therefore the possibility of the tumors being suitable for surgical resection is very low (10-20%). Recently, several improvements have been achieved for the imaging analysis of pancreatic structure, including endoscopic retrograde cholangiopancreatography (ERCP), magnetic resonance cholangiopancreatography (MRCP) and endoscopic ultrasound system (Adamek, H. E., Albert, J., Breer, H., Weitz, M., Schilling, D., and Riemann, J. F. Pancreatic cancer detection with magnetic resonance cholangiopancreatography and endoscopic retrograde cholangiopancreatography: a prospective controlled study. Lancet, 356: 190-193, 2000.). However, even with these procedures, there often exists a difficulty to distinguish pancreatic carcinoma from other disorders such as chronic pancreatitis.

To make matters worse, these methods can usually detect pancreatic tumors that are larger than 5 mm of diameter. Considering the low five-year survival rate (20-30%) of even small, resectable tumors, the current technologies do not have a sensitivity high enough allowing to detect pancreatic carcinoma at “early” stages. To achieve the “cure” of this disorder, it would be necessary to detect the tumors at a bona fide early stage, or carcinoma in situ.

Since pancreas ductal carcinoma (PDC) arises from the epithelial cells of pancreatic duct, a part of carcinoma cells dropped off into pancreatic juice. Investigation of these cells seems to be a promising way to develop a novel means for the sensitive diagnosis of pancreatic carcinoma. Actually, molecular biological analysis of these tumor cells has revealed a variety of genetic alterations in the development of pancreatic carcinoma. The activating point mutations of the K-RAS proto-oncogene has been found in more than 80% of the cases (Kondo, H., Sugano, K., Fukayama, N., Kyogoku, A., Nose, H., Shimada, K., Ohkura, H., Ohtsu, A., Yoshida, S., and Shimosato, Y. Detection of point mutations in the K-ras oncogene at codon 12 in pure pancreatic juice for diagnosis of pancreatic carcinoma. Cancer, 73: 1589-1594, 1994.), and inactivation of p53 tumor-suppressor gene at the similar frequency (Sugano, K., Nakashima, Y., Yamaguchi, K., Fukayama, N., Maekawa, M., Ohkura, H., Kakizoe, T., and Sekiya, T. Sensitive detection of loss of heterozygosity in the TP53 gene in pancreatic adenocarcinoma by fluorescence-based single-strand conformation polymorphism analysis using blunt-end DNA fragments. Genes Chromosomes Cancer, 15: 157-164, 1996.). Other genetic mutations may be found within the genes for p16, DPC4 and DCC (Caldas, C., Hahn, S. A., da Costa, L. T., Redston, M. S., Schutte, M., Seymour, A. B., Weinstein, C. L., Hruban, R. H., Yeo, C. J., and Kern, S. E. Frequent somatic mutations and homozygous deletions of the p16 (MTS1) gene in pancreatic adenocarcinoma. Nat. Genet., 8: 27-32, 1994.; Hahn, S. A., Schutte, M., Hoque, A. T., Moskaluk, C. A., da Costa, L. T., Rozenblum, E., Weinstein, C. L., Fischer, A., Yeo, C. J., Hruban, R. H., and Kern, S. E. DPC4, a candidate tumor suppressor gene at human chromosome 18q21.1. Science, 271: 350-353, 1996.; Hohne, M. W., Halatsch, M. E., Kahl, G. F., and Weinel, R. J. Frequent loss of expression of the potential tumor suppressor gene DCC in ductal pancreatic adenocarcinoma. Cancer Res., 52: 2616-2619, 1992.). However, K-RAS mutations can be also detected in non-malignant pancreatic disorders at a relatively high frequency (Furuya, N., Kawa, S., Akamatsu, T., and Furihata, K. Long-term follow-up of patients with chronic pancreatitis and K-ras gene mutation detected in pancreatic juice. Gastroenterology, 113: 593-598, 1997.). To date, there are no molecular markers proved specific to the carcinoma cells of pancreatic ductal origin.

### DISCLOSURE OF THE INVENTION

DNA microarray enables us to monitor the expression profile of thousands of genes simultaneously (Duggan, D. J., Bittner, M., Chen, Y., Meltzer, P., and Trent, J. M. Expression profiling using cDNA microarrays. Nat. Genet., 21: 10-14, 1999.; Schena, M., Shalon, D., Davis, R. W., and Brown, P. O. Quantitative monitoring of gene expression patterns with a complementary DNA microarray. Science, 270: 467-470, 1995.), and, thus, would be a suitable screening system to identify PDC-specific genes. The high throughput ability of this methodology can become, however, a “double-sided sword”. Without the thoughtful design in the sample preparation or data normalization procedures, DNA microarray experiments yield a large number of pseudo-positive and pseudo-negative results.

In the case of PDC, we suspected that a simple comparison of pancreatic tissues obtained from non-malignant and cancerous cases would be such one. Majority of normal pancreatic tissue is comprised of exocrine and endocrine cells, and proportion of the volume occupied by ductal structure within normal pancreas is very small. In contrast, however, the cancerous tissue is mainly occupied by tumor cells that originate from ductal epithelial cells. Therefore, a comparison between non-malignant and cancer tissues would mainly identify the difference of gene expression profiles between exocrine/endocrine cells and those of ductal cell-origin, not between the normal and transformed cells of the same origin.

It is an object of the present invention to reduce the generation of such false-positive and false-negative results, in the identification of a gene specific for the carcinoma cell of pancreatic ductal-origin. Therefore, the present invention provides a method capable of efficiently identifying a pancreatic ductal carcinoma-specific gene.

Furthermore, it is considered that the pancreatic ductal carcinoma-specific gene being obtainable using the identification method becomes the important target of drug development for the test of the pancreatic ductal carcinoma and the treatment or prevention of the pancreatic ductal carcinoma. Accordingly, it is further object of the present invention to provide a method of testing for pancreatic ductal carcinoma using, as a target, the pancreatic ductal carcinoma-specific gene identified by the above-mentioned method, and a method of screening a pharmaceutical candidate compound for the treatment or prevention of the pancreatic ductal carcinoma.

Surgical resection of PDC at curable stages is hampered by a lack of sensitive and reliable detection methods for PDC. Since DNA microarray makes it possible to monitor the expression profiles of thousands of genes simultaneously, it would be a suitable means to identify novel molecular markers for the clinical diagnosis of PDC. However, although this method seems promising, a simple comparison between normal and cancerous pancreatic tissues yields misleading pseudo-positive data which mainly reflect the different cell-composition within specimens; while normal pancreatic tissues are mainly comprised of endocrine/exocrine cells, cancerous pancreatic tissues are largely occupied by cancer cells of ductal cell-origin. Indeed, our microarray comparison of normal and cancerous tissues has identified the insulin gene as one of the most specific genes to the former. To eliminate such “population-shift” effects, it would be helpful to isolate PDC cells and their origin, normal pancreatic ductal cells, and to directly compare the transcriptome of these purified fractions. Toward this goal, we purified ductal epithelial cells, by the use of affinity column for MUC1, from the pancreatic juice isolated from healthy individuals as well as those with PDC. Microarray analysis among these background-matched samples of 3456 human genes has identified a number of carcinoma-specific genes, such as those for AC133 and carcinoembryonic antigen-related cell adhesion molecule 7 (CEACAM7). Cancer-specific expression of these genes was further confirmed by a quantitative real-time PCR method. Our microarray analysis with purified pancreatic ductal cells has paved a novel way to develop a sensitive detection method for PDC by the use of pancreatic juice which is routinely obtained in clinical conditions.

A pancreatic ductal carcinoma-specific gene can be efficiently identified by utilizing this method, and thereby, it is possible to provide a target that is important for developing a drug for the test of pancreatic ductal carcinoma and the treatment or prevention of pancreatic ductal carcinoma.

<Identification Method of Pancreatic Ductal Carcinoma-Specific Gene>

The present invention provides a method of identifying a pancreatic ductal carcinoma-specific gene. The term “pancreatic ductal carcinoma-specific gene” in the present invention means a gene in which expression changes significantly in a pancreatic ductal carcinoma patient in comparison with a healthy individual. Accordingly, both of a gene specifically expressed in the pancreatic ductal carcinoma patient and a gene specifically expressed in the healthy individual are included in the “pancreatic ductal carcinoma specific gene”. Herein, the term “significant” means that the difference in the expression level of control is 1.5-fold or more, preferably 3-fold or more, and preferably 5-fold or more (for example, 10-fold or more, 20-fold or more, 30-fold or more and 50-fold or more).

In the method of the present invention, first, pancreatic ductal cells are prepared from a pancreatic ductal carcinoma patient and a healthy individual, the gene expression in pancreatic ductal cells prepared from the pancreatic ductal carcinoma patient and the gene expression in pancreatic ductal cells prepared from the healthy individual are detected, the gene expression in the pancreatic ductal cells prepared from the pancreatic ductal carcinoma patient is compared with the gene expression in the pancreatic ductal cells prepared from the healthy individual, and a gene that is specifically expressed in the pancreatic ductal carcinoma patient and a gene that is specifically expressed in the healthy individual are identified.

Techniques of preparing the pancreatic ductal cells from a patient and a healthy individual include, for example, methods of preparing them from pancreatic juice by an affinity column using pancreatic ductal cells-specific protein as an index, but not particularly limited thereto. In this case, as a protein used for the index of pancreatic ductal cells, for example, a MUC1 protein can be preferably utilized. As other techniques of preparing pancreatic ductal cells, there can be also considered a method of taking out the area of cells of interest by laser irradiation under observation by a microscope, for example, a laser capture microdissection (LCM) method. However, it is required to fix and stain tissue for observation by a microscope, and there is a possibility that RNA in cells is damaged through such procedures, and thus the method of using the above-mentioned pancreatic juice is preferred.

Both of transcription and translation are included in the “gene expression” in the present invention. Accordingly, both of detection at a transcription level (mRNA, cDNA) and detection at a translation level (protein) are included in the “detection of the gene expression”.

The detection of gene expression at the transcription level can be measured, for example, by a DNA array method (M. Muramatsu, M. Yamamoto, “Shin-Idenshi-Kogaku Hand Book (New Gene Technology Hand Book)” published by Youdosha Co., 280-284).

In the DNA array method, first, a cDNA sample is prepared from pancreatic ductal cells, the cDNA sample is contacted with a substrate on which an oligonucleotide probe has been fixed, and the hybridization signal of the cDNA sample with the oligonucleotide probe which has been fixed on the substrate is detected.

The preparation of the cDNA sample can be carried out by a method known to those skilled in the art. In the preferable embodiment of preparing the cDNA sample, the extraction of total RNA from the pancreatic ductal cells is first carried out. Existing methods, kits and such can be used for the extraction of the total RNA, so long as they allow to prepare the highly purified total RNA. For example, the total RNA can be extracted using RNA sol B (Teltest Inc., Friendswood, Tex.). Furthermore, the total RNA can be extracted using “Isogen” from Nippon Gene Co., Ltd., after carrying out pretreatment using “RNA later” from Ambion Co. Specifically, the methods may be carried out according to attached protocols thereto. Then, the synthesis of cDNA is carried out using reverse transcriptase using, as a template, the total RNA extracted, and thus, the cDNA sample is prepared. The synthesis of the cDNA sample from the total RNA can be carried out by a method known to those skilled in the art. The cDNA sample prepared is labeled for detection, if necessary. The label substance is not specifically limited so long as it can be detected, and it includes, for example, a fluorescent substance, a radioactive element, and so on. The marker can be carried out by a method that is conducted in general by those skilled in the art (L. Luo et al., Gene expression profiles of laser-captured adjacent neuronal subtypes. Nat Med. 1999, 117-122). For example, a biotin labeled cDNA can be synthesized from an amplified sample RNA (2 μg) using an ExpressChip labeling system (Mergen, San Leandro, Calif.). When the biotin labeled cDNA is used, after hybridization with a DNA array, it is continuously incubated together with streptavidin, an antibody for streptavidin and a Cy3-binding secondary antibody (all obtained from Mergen Co.), and the detection of a hybridization signal and digitalization can be carried out utilizing a GMS 418 array scanner (Affymetrix Co., Santa Clara, Calif.).

The advantage of DNA array technology is that the solution volume of hybridization is very little, and a very complicated target containing cDNA which is derived from the total RNA in cells can be hybridized using the nucleotide probe which has been fixed. In general, DNA array is constituted by thousands of nucleotides printed on a substrate in high density. These DNA's are usually printed on the surface layer of non-porous substrate. The surface layer of the substrate is glass in general, but a porous membrane, for example nitrocellulose membrane, can be used. There are two types for fixation (array) of nucleotides. One is an array in which oligonucleotides developed by Affymetrix Co. is a nucleotide, and another one is a cDNA array mainly developed by Stanford University. In the oligonucleotide array, oligonucleotides are generally synthesized in situ. For example, a photolithographic technique (Affymetrix Co.) and a method of synthesizing oligonucleotides in situ by an ink jet technique (Rosetta Inpharmatics Inc.) for fixing a chemical substance and so on are already known. Either of the techniques can be used for the preparation of the substrate of the present invention.

As the oligonucleotide probes which are fixed on a substrate, the oligonucleotide probes that are specifically hybridized to a human gene are preferred. Oligonucleotide probes of the present invention include synthetic oligonucleotides and cDNA. As the DNA array on which the oligonucleotide probes have been fixed, a commercially available one can be also used, and for example, a micro array (HO-1 to 3, from Mergen) including oligonucleotides corresponding to total 3456 human genes is one preferable example.

The reaction liquid and reaction conditions of hybridization of the cDNA sample with the oligonucleotide probes on a substrate can be fluctuated by various factors such as the length of the nucleotide probes which are fixed on the substrate, but those skilled in the art can set an appropriate condition to carry out the hybridization reaction.

The method of collectively detecting the gene expression at transcription level other than the DNA array method includes a cDNA subtraction cloning method. This technique has a defect that the expression level cannot be quantitatively evaluated compared with the DNA array method, but it has an advantage that an unknown gene can be also cloned uniformly. A commercially available kit, for example, the “PCR-Select cDNA subtraction kit (#K1804-1)” (Clontech Co.) can be used for the method.

Furthermore, in the present invention, it can be also considered to detect the expression of a gene at the translation level. In this case, a protein sample is first prepared from pancreatic ductal cells, and the expression of respective proteins is detected. As the detection method of proteins, methods well known to those skilled in the art, for example, a SDS polyacrylamide electrophoresis method, a two-dimensional electrophoresis method, and so on can be used.

Following the above-mentioned detection, the gene expression in the pancreatic ductal cells prepared from a pancreatic ductal carcinoma patient is compared with the gene expression in the pancreatic ductal cells prepared from a healthy individual. As the result of the comparison, a gene having significantly high or low expression level in the pancreatic ductal carcinoma patient can be identified as a pancreatic ductal carcinoma-specific gene in comparison with a case in the healthy individual.

<Method of Examination>

The present invention also provides a method of testing for the pancreatic ductal carcinoma. One embodiment of the method of testing for the pancreatic ductal carcinoma of the present invention is to use the expression abnormality of the pancreatic ductal carcinoma-specific gene identified by the above-mentioned method, as an index.

In the examination, first, a tissue or cells are prepared from a subject, the expression of the pancreatic ductal carcinoma-specific gene which is identified by the above-mentioned identification method of the present invention is detected in the tissue or cells, and the expression of the detected pancreatic ductal carcinoma-specific gene is compared with the expression of the gene in control tissue or cells.

As the tissue or cells prepared from a subject, the pancreatic ductal cells can be preferably used. The preparation of the pancreatic ductal cells is as mentioned above. Pancreatic ductal carcinoma-specific genes which is subjective to detection, for example, include receptor type protein tyrosine phosphatase U (PTPRU; Genbank accession No. U73727) whose specificity to the pancreatic ductal carcinoma have been identified by the present inventors, membrane ingredient, chromosome 1, surface marker 1 (M1S1; X77753), matrix metalloproteinase 9 (MMP9; J05070), AC133 (AF027208), protein phosphatase 2, regulatory subunit B, α-iso type (PPP2R5A; L42373), properdin factor B (BF; L15702), amyloid P ingredient, serum (APCS; X04608), and a gene of CEACAM7 (X98311), additionally the genes described in Tables 1 to 8. Since AC133, CEACAM7, SOD2 and HSP105 have very high specificity for the pancreatic ductal carcinoma, it is the particularly preferable index of the pancreatic ductal carcinoma.

Pancreatic ductal carcinoma-specific genes for subjects of test can be combinations of the genes mentioned above. A combination SOD2 and HSP105 is preferable for the present invention. “2e(act−marker gene)×1000” in tabeles3 to 8 and 11 to 14 is a preferable index to test for the pancreatic ductal carcinoma. If the value of the index of a subject is more than one (for example, more than two, three, four, or five), the subject is suspected of having pancreatic ductal carcinoma (Tables9 and 10). For example, if the value of “2e(act−SOD2)×1000” is more than five or the value of “2e(act−HSP105)×1000” is more than one, the subject is strongly suspected of having pancreatic ductal carcinoma (Tables13 and 14).

Both of transcription and translation are included in the “gene expression” in the present invention. Accordingly, both of detection at the transcription level (mRNA, cDNA) and detection at the translation level (protein) are included in the “detection of the gene expression”.

In the detection of the gene expression at the transcription level, a RNA sample is prepared from the tissue or cells which are prepared from a subject, and the RNA level of the pancreatic ductal carcinoma-specific gene which is contained in the RNA sample is measured. Such methods can be exemplified by Northern blotting using a probe hybridized to the transcription product of the pancreatic ductal carcinoma-specific gene, an RT-PCR method using a primer hybridized to the transcription product of the pancreatic ductal carcinoma-specific gene, or a PCR method using a primer hybridized to cDNA prepared from the transcription product of the pancreatic ductal carcinoma-specific gene, and so on. The preparation of the RNA sample and cDNA sample is as mentioned above.

In the detection of the gene expression at the translation level, a protein sample is prepared from the tissue or cells that are prepared from a subject, and the amount of the protein encoded by the pancreatic ductal carcinoma-specific gene which is contained in the protein sample, is measured. Such method can be exemplified by a SDS polyacrylamide electrophoresis method, a western blotting method using antibody which is bonded to the protein encoded by the pancreatic ductal carcinoma-specific gene, a dot blotting method, an immunoprecipitation method, an enzyme linkage immunoassay method (ELISA), and an immunofluorescence method. As the control for the detection of the expression of the pancreatic ductal carcinoma-specific gene in a subject, the expression level of the pancreatic ductal carcinoma-specific gene in a healthy individual is usually used.

It is considered that the gene specifically expressed in a pancreatic ductal carcinoma patient is involved in the onset of the pancreatic ductal carcinoma. On the other hand, it is considered that a gene not specifically expressed in a pancreatic ductal carcinoma patient is involved in suppressing the onset of the pancreatic ductal carcinoma. Accordingly, when the gene specifically expressed in a pancreatic ductal carcinoma patient as the pancreatic ductal carcinoma-specific gene is used as a target, a subject is judged to have a possibility having the pancreatic ductal carcinoma if the expression level of the pancreatic ductal carcinoma-specific gene in the subject is significantly increased in comparison with that of a control. When the gene not specifically expressed in a pancreatic ductal carcinoma patient as the pancreatic ductal carcinoma-specific gene is used as a target, a subject is judged to have a possibility having the pancreatic ductal carcinoma if the expression level of the pancreatic ductal carcinoma-specific gene in the subject is significantly low in comparison with that of a control.

Another embodiment of the examination of the present invention is a method of using the expression abnormality of the pancreatic ductal carcinoma-specific gene that has been identified by the above-mentioned method, or the genetic polymorphism or mutation which causes the activity abnormality of protein encoded by the gene, as an index.

Herein, the term “mutation” indicates the variation of an amino acid in an amino acid sequence or the variation of a nucleotide in a nucleotide sequence (i.e. substitution, deletion, addition or insertion of one or more amino acids or nucleotides). In addition, the “genetic polymorphism” is genetically defined in general as the variation of a certain nucleotide in a gene which exists at a frequency of 1% or more in the population. However, the “genetic polymorphism” in the present invention is not limited by the definition, and includes also the variation of a nucleotide at less than 1%, in the “polymorphism”. Accordingly, the “mutation” and “genetic polymorphism” in the present specification is not strictly discriminated, and means the variation of an amino acid in an amino acid sequence or the variation of a nucleotide in a nucleotide sequence, using both as an integrated one (as a phrase of “genetic polymorphism or mutation”).

The genetic polymorphism or mutation in a subject is not specifically limited to its kind, number and site so long as it causes the expression abnormality of the pancreatic ductal carcinoma-specific gene, or the activity abnormality of protein is encoded by the gene.

The detection of the genetic polymorphism or mutation can be carried out, for example, by directly determining the nucleotide sequence of the pancreatic ductal carcinoma-specific gene in a subject. In the method, a DNA sample is first prepared from a subject. The DNA sample can be prepared based on chromosome DNA or RNA, which is sampled by the pancreatic juice, blood, skin, tunica mucosa oris, and tissue or cells derived from surgically removed or excised pancreas. Then, a DNA containing the region of the pancreatic ductal carcinoma-specific gene is isolated. The isolation of the DNA can be carried out by PCR or such which uses the chromosome DNA or RNA as a template, using a primer hybridized to a DNA comprising the region of the pancreatic ductal carcinoma-specific gene. Then, the nucleotide sequence of isolated DNA is determined. The determination of the nucleotide sequence of isolated DNA can be carried out by a method known to those skilled in the art. When the above-mentioned genetic polymorphism and mutation exist in the nucleotide sequence of determined DNA, the subject is judged to have a possibility having the pancreatic ductal carcinoma.

The examination of the present invention can be carried out by various methods to detect polymorphism or mutation, other than a method of directly determining the nucleotide sequence of DNA from a subject as described above.

In one embodiment, first, a DNA sample is prepared from a subject. Then, the DNA sample prepared is digested by restriction enzyme. Resulting DNA fragments are separated in accordance with its size. Then, the size of the detected DNA fragment is compared with that of a control. Alternatively, in another embodiment, first, a DNA sample is prepared from a subject. Then, a DNA containing a expression control region of the pancreatic ductal carcinoma-specific gene is amplified. Furthermore, the amplified DNA is digested by restriction enzyme. Then, DNA fragments are separated in accordance with its size. Then, the size of the detected DNA fragment is compared with that of a control.

The above method may, for example, utilize the Restriction Fragment Length Polymorphism/RFLP, the PCR-RFLP method, and so on. Specifically, when a mutation exists in the recognition site of a restriction enzyme, or when insertion(s) or deletion(s) of nucleotide(s) exists in a DNA fragment generated by a restriction enzyme treatment, the fragments generated after the restriction enzyme treatment differ in terms of size from those of controls. The portion containing the mutation is amplified by PCR, and then, is treated with respective restriction enzymes to detect the mutation as a difference in the mobility of bands by electrophoresis. Alternatively, the presence or absence of a mutation on the chromosomal DNA can be detected by treating the chromosomal DNA with these restriction enzymes, subjecting the fragments to electrophoresis, and then, carrying out Southern blotting with a probe DNA hybridized to the pancreatic ductal carcinoma-specific gene. The restriction enzymes to be used can be appropriately selected in accordance with respective mutations. The Southern blotting can be conducted not only on the genomic DNA but also on cDNAs directly digested with restriction enzymes, wherein the cDNAs are synthesized by a reverse transcriptase from RNAs prepared from subjects. Alternatively, after amplifying a DNA containing a expression control region of the pancreatic ductal carcinoma-specific gene by PCR using the cDNA as a template, the cDNAs can be digested with restriction enzymes, and the difference of mobility can be examined.

Furthermore, in another method, a DNA sample is first prepared from a subject. Then, a DNA containing a region of the pancreatic ductal carcinoma-specific gene is amplified. Furthermore, the amplified DNA is dissociated to single strand DNA. The single strand DNA dissociated is separated on non-degenerating gel. The mobility on the gel of the single strand DNA separated is compared with that of a control.

The above method may, for example, utilize the PCR-SSCP (single-strand conformation polymorphism) method (“Cloning and polymerase chain reaction-single-strand conformation polymorphism analysis of anonymous Alu repeats on chromosome 11.” Genomics 1992, Jan. 1, 12(1): 139-146; “Detection of p53 gene mutations in human brain tumors by single-strand conformation polymorphism analysis of polymerase chain reaction products.” Oncogene 1991, Aug. 1; 6(8): 1313-1318; “Multiple fluorescence-based PCR-SSCP analysis with postlabeling.” PCR Methods Appl. 1995, Apr. 1; 4(5): 275-282). This method is particularly preferable for screening many DNA samples, since it has advantages such as: comparative simplicity of operation; small amount of required test sample; and so on. The principle of the method is as follows. A single stranded DNA dissociated from a double-stranded DNA fragment forms a unique higher conformation, depending on respective nucleotide sequence. After electrophoresis on a polyacrylamide gel without a denaturant, complementary single-stranded DNAs having the same chain length of the dissociated DNA strand shift to different positions in accordance with the difference of the respective higher conformations. The conformation of a single-stranded DNA changes even by a substitution of one base, which change results in a different mobility on polyacrylamide gel electrophoresis. Accordingly, the presence of a mutation in a DNA fragment due to even a single point mutation, deletion, insertion, and such can be determined by detecting the changes in the mobility.

More specifically, a DNA containing a region of the pancreatic ductal carcinoma-specific gene is first amplified by PCR or such. Preferably, a length of about 200 to 400 bp is amplified. PCR can be carried out by those skilled in the art, appropriately selecting a reaction condition and such. The amplified DNA products can be labeled by PCR using primers which are labeled with isotopes such as ³²P; fluorescent dyes; biotin; and so on, or by adding into the PCR solution substrate nucleotides which are labeled with isotopes such as ³²P; fluorescent dyes; biotin; and so on. Alternatively, the labeling of the DNA fragments can be carried out by adding after PCR substrate nucleotides labeled with isotopes, such as ³²P; fluorescent dyes; biotin; and so on, to the amplified DNA fragment using the Klenow enzyme and such. Then, the obtained labeled DNA fragments are denatured by heating and the like, to be subjected to electrophoresis on a polyacrylamide gel without a denaturant, such as urea. The condition for separating DNA fragments in the electrophoresis can be improved by adding appropriate amounts (about 5 to 10%) of glycerol to the polyacrylamide gel. Further, although the condition for electrophoresis varies depending on the character of respective DNA fragments, it is usually carried out at room temperature (20 to 25° C.). In the event a preferable separation is not achieved at this temperature, a temperature to achieve the optimum mobility may be selected from temperatures between 4 to 30° C. After the electrophoresis, the mobility of the DNA fragments is detected by autoradiography with X-ray films, scanner for detecting fluorescence, and the like, to analyze the result. When a band with different mobility is detected, the presence of a mutation can be confirmed by directly excising the band from the gel, amplifying it again by PCR, and directly sequencing the amplified fragment. Further, without using labeled DNAs, the bands can be also detected by staining the gel after electrophoresis with ethidium bromide, silver, and such.

Furthermore, in an alternative method, a DNA sample is first prepared from a subject. Then, a DNA containing a region of the pancreatic ductal carcinoma-specific gene is amplified. Furthermore, the amplified DNA is separated on a gel in which the concentration of a DNA denaturant is gradually enhanced. Then, the mobility of the DNA separated on the gel is compared with that of a control.

As the method of example, a denaturant gradient gel electrophoresis method (DGGE method) and such can be exemplified. The DGGE method is a method by which the mixture of DNA fragments is migrated in a denaturant gradient polyacrylamide gel and the DNA fragments are separated by the difference of respective instabilities. When unstable DNA fragments having miss match are moved to a portion with a certain denaturant concentration in the gel, the DNA sequence around the miss match is partially dissociated to a single strand because of its instability. The mobility of and differentiated from the mobility of a completely double-strand DNA having no dissociated portion, therefore both can be separated. Specifically, the DNA containing the region of the pancreatic ductal carcinoma-specific gene is amplified by the PCR method and such using the primer of the present invention and the like, electrophoresed in a polyacrylamide gel in which the concentration of a denaturant such as urea is gradually enhanced in accordance with movement, and compared with a control. In case of the DNA fragments in which mutation exists, since the DNA fragments become a single strand at a position of lower denaturant concentration, and the mobility becomes extremely slow, the presence or absence of the mutation can be determined by detecting the difference in the mobility.

An Allele Specific Oligonucleotide (ASO) hybridization method can be utilized for detecting mutation only at a specific position, other than the above-mentioned methods. When an oligonucleotide containing a nucleotide sequence in which mutation is considered to exist is prepared, and a sample DNA is hybridized to this. If the mutation exists, the efficiency of hybrid formation is reduced. It can be detected by the southern blotting method, a method of utilizing a property of quenching by intercalating a specific fluorescent reagent into the gap of a hybrid, and such. In addition, detection using a ribonuclease A miss match fragmentation method can be carried out. Specifically, the DNA containing the region of the pancreatic ductal carcinoma-specific gene is amplified by the PCR method or such, and the amplified product is hybridized to labeled RNA which is prepared from the cDNA of the pancreatic ductal carcinoma-specific gene and such incorporated in plasmid vector and the like. Since hybrid becomes a single strand conformation at a portion where mutation exists, the portion is digested by ribonuclease A, and the presence of the mutation can be determined by detecting this by an autoradiography and such.

As a result of detection by the detection method described above, a subject is judged to have a possibility having the pancreatic ductal carcinoma when the subject has the expression abnormality of the pancreatic ductal carcinoma-specific gene, or the genetic polymorphism or mutation which causes the activity abnormality of protein encoded by the gene.

<Test Agent>

The present invention also provides an agent for testing for the pancreatic ductal carcinoma. One embodiment of the test agent of the present invention contains, as an effective ingredient, oligonucleotide which is specifically hybridized to the transcription product of the pancreatic ductal carcinoma-specific gene. These can be used for testing for the pancreatic ductal carcinoma in which the above-mentioned gene expression is used as an index, or for noma in which the genetic polymorphism or mutation is used as an index.

Herein, the “specifically hybridized” means that cross hybridization with a DNA encoding for other protein is not significantly generated under conditions of usual hybridization, and preferably under conditions of stringent hybridization (for example, conditions which is described in Sambrook et al., “Molecular Cloning”, Cold Spring Harbour Laboratory Press, New York, USA, the second edition, 1989). When the specific hybridization is possible, it is unnecessary that the oligonucleotide is completely complimentary to the nucleotide sequence of the pancreatic ductal carcinoma-specific gene to be detected.

The oligonucleotide can be used as a probe or a primer in the above-mentioned examination of the present invention. When the oligonucleotide is used as the primer, the length is usually 15 bp to 100 bp, and preferably 17 bp to 30 bp. The primer is not specifically limited so long as it amplifies at least a portion of the transcription product of the pancreatic ductal carcinoma-specific gene.

Furthermore, when the above-mentioned oligonucleotide is used as the probe, the probe is not specifically limited so long as it is specifically hybridized to at least a portion of the transcription product of the pancreatic ductal carcinoma-specific gene. The probe may be synthetic oligonucleotide, and has usually a chain length of at least 15 bp or more.

The oligonucleotide of the present invention can be prepared, for example, by a commercially available oligonucleotide synthesizer. The probe can be also prepared as a double strand DNA fragment which is obtained by restriction enzyme treatment and such.

When the oligonucleotide of the present invention is used as a probe, it is preferably labeled as necessary. As the labeling method can be exemplified by a method of labeling by phosphorylating the 5′ terminal of oligonucleotide with ³²P using T4 polynucleotide kinase, and a method of incorporating a substrate nucleotide labeled by isotopes such as ³²P, a fluorescent dye, or biotin using random hexamer oligonucleotide and such as a primer, using DNA polymerase such as Klenow enzyme (such as a random prime method).

In another embodiment of the test agent of the present invention, an antibody which is combined with the protein encoded by the pancreatic ductal carcinoma-specific gene is used as an effective ingredient. While the antibody is not specifically limited so long as it is an antibody which can be used for the test, it can be exemplified by polyclonal antibody and monoclonal antibody. The antibody is labeled in accordance with requirement.

The antibody can be prepared by methods known to those skilled in the art. The polyclonal antibody can be obtained, for example, in the following manner. A small animal such as a rabbit is immunized with a natural-occurring protein encoded by the pancreatic ductal carcinoma-specific gene, a recombinant protein expressed in microorganisms such as E. coli as fusion protein with GST, or partial peptide thereof, to obtain serum. This is purified by, for example, ammonium sulfate sedimentation; protein A or protein G column; DEAE ion exchange chromatography; or an affinity column which has coupled a protein encoded by the pancreatic ductal carcinoma-specific gene or synthetic peptide thereof, to prepare a polyclonal antibody. Alternatively, in case of the monoclonal antibody, for example, a small animal such as a mouse is immunized with a protein encoded by the pancreatic ductal carcinoma-specific gene or partial peptide thereof, the spleen is enucleated from the mouse, followed by triturating to separate cells, the cells are fused with mouse myeloma cells using a reagent such as a polyethylene glycol, and clone which produces antibody which is coupled with the protein encoded by the pancreatic ductal carcinoma-specific gene is selected from resulting fused cells (hybridomas). Then, the obtained hybridomas are transplanted in mouse abdominal cavity, the ascites is collected from the mouse, and the obtained monoclonal antibody is purified by, for example, ammonium sulfate sedimentation, protein A or protein G column, DEAE ion exchange chromatography, an affinity column and such which has coupled a protein encoded by the pancreatic ductal carcinoma-specific gene or synthetic peptide, to prepare the monoclonal antibody.

In the above-mentioned test agent, for example, sterilized water, physiological saline, vegetable oil, a surfactant, lipid, a dissolution aid, a buffer, a protein stabilizer (BSA, gelatin, etc.), a preservative, and so on may be mixed other than oligonucleotide which is an effective ingredient, and antibody, if necessary.

<Identification Method of Pharmaceutical Candidate Compound>

The present invention also provides a method for identifying a pharmaceutical candidate compound for the treatment or prevention of the pancreatic ductal carcinoma.

One embodiment of the identification method of a pharmaceutical candidate compound of the present invention is a method of using the expression of the pancreatic ductal carcinoma-specific gene, as an index.

In the present method, a test compound is first administered in or contacted with a test animal or test cells, and then, the expression of the pancreatic ductal carcinoma-specific gene in the test animal or test cells is detected.

Test animals used include, for example, a monkey, a mouse, a rat, a caw, a pig, and a dog. Origin of the test animals includes, for example, a human, a monkey, a mouse, a rat, a caw, a pig, and a dog. However, they are not limited thereto. As the “test cells”, for example, the pancreatic ductal cells can be preferably used.

Test compounds used in the present method include, for example, single compounds such as a natural compound, an organic compound, an inorganic compound, protein and peptide, expression products of compound library and gene library, a cell extract, a cell cultured supernatant, a product of fermented microbe, an extract of marine organism, and a plant extract.

As the “administration” of the test compound to a test animal, for example, blood administration by injection, oral administration, percutaneous administration and the like are considered. Further, the “contacting” the test compound to the test cells is usually carried out by adding a compound to be tested to the culture medium of the test cells. However, the techniques of “administration” and “contacting” according to the present method are not limited thereto. When a test compound is a protein and such, the “contacting” can be carried out by introducing a DNA vector which expresses the protein, in the cells.

The detection of the gene expression in the present method includes both of the detection of transcription level and the detection of translation level. The measurement of the transcription level can be carried out by methods known to those skilled in the art. For example, mRNA is extracted from test cells according to standard methods, and the transcription level of the gene can be determined by conducting Northern hybridization that uses the mRNA as a template, or the RT-PCR method. The transcription level of the gene can be also measured using DNA array techniques. Further, a protein fraction is collected from test cells, and the translation level can be also determined by detecting the expression of a target protein by an electrophoresis method such as SDS-PAGE. Furthermore, the translation level can be also determined by detecting the expression of the protein by conducting Western blotting using an antibody for a target protein. The antibody used for detection is not specifically limited, and, for example, a monoclonal antibody, a polyclonal antibody or a fragment thereof can be utilized.

It is considered that a gene specifically expressed in a pancreatic ductal carcinoma patient is involved in the onset of the pancreatic ductal carcinoma. On the other hand, it is considered that a gene not specifically expressed in a pancreatic ductal carcinoma patient is involved in the suppression of the onset of the pancreatic ductal carcinoma. Accordingly, as a result of the detection, in case of using, as a target, a gene specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the expression level of the gene is significantly reduced by administration of the test compound. On the other hand, in case of using, as a target, a gene not specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the expression level of the gene is significantly increased by administration of the test compound.

In another embodiment of the identification method of the pharmaceutical candidate compound in the present invention, the expression of the pancreatic ductal carcinoma-specific gene is detected utilizing a reporter system.

In the present method, a test compound is first administered in, or contacted with a test animal or test cells having a reporter gene operably linked with the expression control region (promoter region) of the pancreatic ductal carcinoma-specific gene. Herein, the term “operably linked” means that the expression control region is coupled with the reporter gene so that the expression of the reporter gene is induced by coupling the transcription factor with the expression control region of the pancreatic ductal carcinoma-specific gene. Accordingly, when the reporter gene is coupled with other gene and the fusion protein coupled with other gene product is formed, it is included in the meaning of the above-mentioned term “operably linked” so long as the expression of the fusion protein is induced by coupling the transcription factor with the expression control region.

The reporter gene used in the present invention is not specifically limited so long as the expression can be detected, and includes, for example, CAT gene, lacZ gene, luciferase gene, and GFP gene.

A vector having the reporter gene operably linked with the expression control region (promoter region) of the pancreatic ductal carcinoma-specific gene can be prepared by methods well known to those skilled in the art. The introduction of the vector into cells can be carried out by general methods, such as a calcium phosphate precipitation method, an electroporation method, a lipofectamine method, and a micro injection method. The term “having the reporter gene operably linked with the expression control region of the pancreatic ductal carcinoma-specific gene” includes also a state in which the construct is inserted in chromosome. The insertion of a DNA construct into chromosome can be carried out by methods usually used by those skilled in the art, for example, a method of introducing a gene utilizing homologous recombination.

As the “administration” of the test compound to a test animal, for example, blood administration by injection, oral administration, percutaneous administration and such are considered. Further, the “contacting” the test compound to the test cells is usually carried out by adding a compound to be tested to the culture medium of the test cells. However, the techniques of the “administration” and the “contacting” according to the present method are not limited thereto. When the compound to be tested is protein and such, the “contacting” can be carried out by introducing a DNA vector which expresses the protein in the cells.

The expression level of the reporter gene in the present method can be measured by methods known to those skilled in the art, in accordance with the kind of the reporter gene. For example, when the reporter gene is a CAT gene, the expression level of the reporter gene can be measured by detecting the acetylation of the gene product by chloramphenicol. The expression level of the reporter gene can be measured by detecting the coloring of a dye compound by the catalyst action of the gene expression product when the reporter gene is a lacZ gene; by detecting the fluorescence of a fluorescent compound by the catalyst action of the gene expression product when it is a luciferase gene; and by detecting the fluorescence by GFP protein when it is a GFP gene.

As a result of the detection, in case of using, as a target, a gene specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the expression level of a reporter gene is significantly reduced by administration of the test compound. On the other hand, in case of using, as a target, a gene not specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the expression level of a reporter gene is significantly increased by administration of the test compound.

In another embodiment of the identification method of the pharmaceutical candidate compound in the present invention, it is a method of using the activity of protein encoded by the pancreatic ductal carcinoma-specific gene, as an index. In this method, the activity of the protein encoded by the pancreatic ductal carcinoma-specific gene is detected by contacting a test compound with the protein.

The protein encoded by the pancreatic ductal carcinoma-specific gene is not specifically limited to its forms, so long as its activity can be detected. The protein may be, for example, a purified form, a form expressed in cells or on cell surface, a form as the cell membrane fraction of the cells, or a form bonded to an affinity column.

The detection of the activity of protein can differ in accordance with the kind of the protein. For example, PTPRU has an activity of removing phosphoric acid from the phosphorylated tyrosine residue of substrate protein; MMP9 has activity as protease; protein phosphatase 2 has activity of removing phosphoric acid from either the phosphorylated serine residue or the phosphorylated threonine residue of substrate protein; and SOD2 has an activity of deactivating a free radical ion which is produced in cells. These activities of the PTPRU, MMP9 and protein phosphatase 2 can be also detected by utilizing a commercially available kit for measuring activities. Specifically, refer to a literature (J. Report, Fertil., 97:347-351, 1993) with respect to the detection of the activity of SOD2.

It is considered that a gene specifically expressed in a pancreatic ductal carcinoma patient is involved in the onset of the pancreatic ductal carcinoma. On the other hand, it is considered that a gene not specifically expressed in a pancreatic ductal carcinoma patient is involved in the suppression of the onset of the pancreatic ductal carcinoma. Accordingly, as a result of the detection, in case of using, as a target, a protein encoded by a gene specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the activity of the protein is reduced by administration of the test compound. On the other hand, in case of using, as a target, a protein encoded by a gene not specifically expressed in a pancreatic ductal carcinoma patient as the pancreatic ductal carcinoma-specific gene, it is judged that the test compound is a pharmaceutical candidate compound for the treatment or prevention of the pancreatic ductal carcinoma if the activity of the protein is increased by administration of the test compound.

### BRIEF DESCRIPTION OF THE DRAWINGS

FIG. 1 Picture: (A) An aliquot of pancreatic juice obtained from an individual with PDC carcinoma was subjected to Cytospin, followed by Wright-Giemsa staining (X 100). In addition to the cells of epithelial origin, both of red blood cells and neutrophils (arrowhead) can be recognized. (B) The eluent from MUC1-affinity column was also stained (X 200). Note the cancer-specific aberrant phenotype (large nuclei with fine chromatin structure) in some cells.

FIG. 2 (A) Comparison of the expression level for 3456 human genes between normal and cancerous pancreatic tissues. The median value of the expression level was calculated for each gene within the 2 PDC tissues, and was compared with that in a normal pancreas tissue. Each line corresponds to a single gene on the array, and shown color-coded according to the expression level in the normal tissue with the color-scheme indicated at the right. (B) Expression profiles of 3456 genes were compared between one normal pancreatic tissue and two MUC1⁺ normal ductal cells as in (A).

FIG. 3 Picture: (A) Hierarchical clustering of 3456 genes based on their expression profiles in tissue samples from one normal individual (NT), two cancer patients (CT), and in MUC1⁺ ductal cells obtained from two normal individuals (ND) and three cancer patients (CD). Each column represents a single gene on the microarray, and each row corresponds to a different patient (or normal) sample. The normalized fluorescence intensity for each gene is shown color-coded as indicated in FIG. 2A. (B) On the basis of the transcriptomes shown in A, two-way clustering analysis was performed to assess statistically the similarity of the samples from the different subjects and to generate a subject dendrogram.

FIG. 4 Picture: (A) The mean expression value of each gene was calculated for the cancerous pancreatic tissue samples (CT) and MUC1⁺ pancreatic ductal cells obtained from normal individuals (ND) or cancer patients (CD). These data, together with the expression level in a normal pancreatic tissue (NT), were used to generate a dendrogram, or “average tree”, with a color-scheme shown in FIG. 2A. (B) From the average tree, genes were selected whose mean level of expression was specifically increased in the CD specimens, and was subjected to the clustering analysis. Each row corresponds to a single gene, with the columns indicating the corresponding expression level in different samples. The gene names, accession numbers as well as their expression intensities are available through the web site of Cancer Science.

FIG. 5 Quantitation of AC133 and CEACAM7 transcripts in MUC1⁺ ductal cells. Complementary DNA prepared from the ductal cells of 8 normal individuals and 10 PDC patients was subjected to real-time PCR with primers specific for AC133 (A) or CEACAM7 (B) or β-actin genes. The ratio of the abundance of the target transcripts to that of β-actin mRNA was calculated as 2ⁿ, where n is the CT value for β-actin cDNA minus the CT value of the target cDNA.

FIG. 6. Quantitation of SOD2 transcripts in MUC1⁺ ductal cells.

Complementary DNA prepared from the ductal cells of 28 pancreatic cancer patients, 16 benign tumor patients, 4 chronic pancreatitis patients and 12 normal individuals was subjected to real-time PCR with primers specific for SOD2 or β-actin genes. The ratio of the abundance of the SOD2 transcripts to that of β-actin mRNA was calculated as 2ⁿ, where n is the C_{T} value for β-actin cDNA minus the C_{T} value of the target cDNA.

### BEST MODE FOR CARRYING OUT THE INVENTION

Herein below, the present invention will be specifically described using examples, however, it is not to be construed as being limited thereto.

### EXAMPLE 1

### Purification of Ductal Cells from Pancreatic Juice

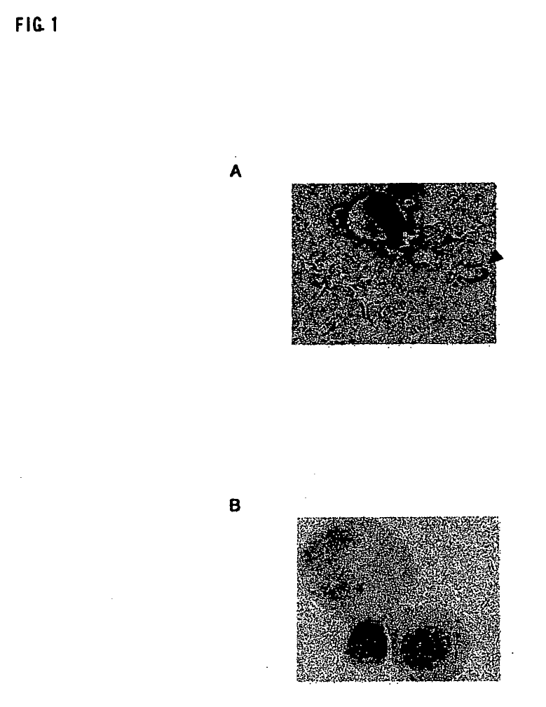

Pancreatic juice contains various types of cells including pancreatic ductal cells, erythrocytes, neutrophils, and lymphocytes (FIG. 1A). Since proportions of these components within the juice significantly vary from patient to patient, a purification step for ductal cells would be required for reliable analyses. Both of normal and cancer-derived pancreatic ductal cells are known to express several mucins. Among them, MUC1 is expressed commonly on normal and cancer ductal cells, whereas other mucins like MUC3 and MUC5 are differentially expressed in a disease-dependent manner (Balague, C., Audie, J. P., Porchet, N., and Real, F. X. In situ hybridization shows distinct patterns of mucin gene expression in normal, benign, and malignant pancreas tissues. Gastroenterology, 109: 953-964, 1995.; Terada, T., Ohta, T., Sasaki, M., Nakamura, Y., and Kim, Y. S. Expression of MUC apomucins in normal pancreas and pancreatic tumours. J. Pathol., 180: 160-165, 1996.). We, hence, chose MUC1 in this study as a common surface marker for pancreatic ductal cells, and developed an affinity purification system for MUC1 by the use of magnetic bead separation column. Specifically, the procedure for purification of ductal cells is shown as follows.

From the individuals subjected to ERCP and to the collection of pancreatic juice for cytological examination, those gave informed consent participated in this study. Diagnosis of the patients was confirmed by the combination of the results with ERCP, cytological examination of pancreatic juice, abdominal CT, serum level of CA19-9 and the follow-up observation of the patients. About one-third of the pancreatic juice was used to purify MUC1⁺-ductal cells as follows. Cells were collected from the pancreatic juice by centrifugation, and re-suspended into 1 ml of MACS-binding buffer (phosphate-buffered saline supplemented with 3% fetal bovine serum and 2 mM EDTA). The cells were then reacted with 0.5 μg of anti-MUC1 antibody (Novocastra Laboratories, Newcastle upon Tyne, UK) at 4° C. for 30 min, washed with the MACS-binding buffer, and mixed with anti-mouse IgG MACS MicroBeads (Miltenyi Biotec, Auburn, Calif.). The cells/MicroBeads mixture was then subjected to chromatography on miniMACS magnetic cell separation columns (Miltenyi Biotec) according to the manufacturer's protocol. The eluted MUC1⁺ cells were divided into aliquots and stored at −80° C. Portions of the unfractionated cells as well as MUC1⁺ cells of each individual were stained with Wright-Giemsa solution to examine the purity of the ductal cell-enriched fractions.

As shown in FIG. 1B, the eluents from the column consisted of the cells with epithelial shape.

### EXAMPLE 2

### The necessity of BAMP Screening for Pancreatic Carcinoma

Previous studies to identify the genes specific to PDC have often compared the gene expression profiles of normal and cancerous pancreatic tissues. However, as discussed in INTRODUCTION, this may result in the identification of genes that are differentially expressed between exocrine/endocrine and ductal cells. To clarify this issue, we first compared the transcriptomes between surgically resected normal (n=1) and cancerous (n=2) pancreatic tissues by using oligonucleotide microarray.

Total RNA was extracted from the MUC1+cell preparations with the use of RNAzol B (Tel-Test Inc., Friendswood, Tex.), and a portion (20 μg) of the RNA was subjected to mRNA amplification with T7 RNA polymerase according to the method of van Gelder et al. (Van Gelder, R. N., von Zastrow, M. E., Yool, A., Dement, W. C., Barchas, J. D., and Eberwine, J. H. Amplified RNA synthesized from limited quantities of heterogeneous cDNA. Proc. Natl. Acad. Sci. USA, 87: 1663-1667, 1990.). Biotin-labeled cRNA was then synthesized from the amplified sample RNA (2 μg) with the use of the ExpressChip labeling system (Mergen, San Leandro, Calif.), and was allowed to hybridize with microarrays (HO-1˜3; Mergen) that contain oligonucleotides corresponding to a total of 3,456 human genes (the gene list can be obtained through its website, http://www.mergen-ltd.com/). The microarrays were then incubated consecutively with streptavidin, antibodies to streptavidin, and Cy3-conjugated secondary antibodies (all from Mergen). Detection and digitization of hybridization signals was performed with a GMS 418 array scanner (Affymetrix, Santa Clara, Calif.).

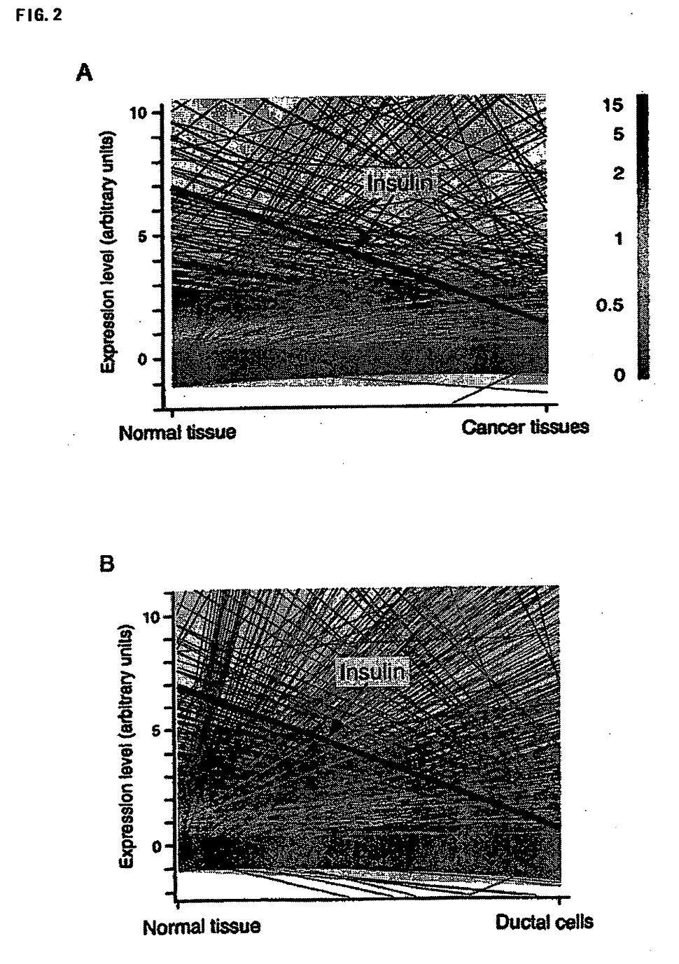

The digitized expression intensities for the 3456 human genes were normalized relative to the median expression level of all genes in each hybridization, and, in the case of cancer tissues, the average expression value for every gene in the two specimens was further calculated. Statistical analysis of the data was performed with GeneSpring 4.0 software (Silicon Genetics, Redwood, Calif.). The expression level of every gene was then compared between the normal and the cancer tissues (FIG. 2A). Interestingly, one of the most specific genes to the normal pancreatic tissue was that for insulin when compared to cancerous one. Since insulin is expressed only in the Langerhans islets, this result may reflect the difference in the proportion of endocrine cells between the samples, not the difference in the number of insulin transcripts per cell between normal and cancer cells.

We also prepared MUC1⁺ ductal cells from two individuals who were revealed, by pathological examination, not to carry PDC. DNA microarray analysis of these specimens and comparison of the data between these normal ductal cells and normal tissue section also indicated that the gene for insulin was one of the most differentially expressed genes between the two groups (FIG. 2B). Furthermore, it was apparent that expression intensities of many genes were distinct between the tissue section and the purified ductal cells.

Since the proportion of cells with ductal origin should strongly increase in the cancerous tissue compared to that in normal pancreatic one, these data collectively support our prediction that a mere comparison of surgically resected specimens between normal and cancerous tissues from pancreas is not a good approach to identify transformation-related genes for the ductal cell lineage.

### EXAMPLE 3

### Expression Profiles of Ductal Cells Obtained from Pancreatic Juice

To identify potential molecular markers specific to PDC, one of the ideal strategy would be to compare the transcriptomes of ductal cells in the pancreatic juice obtained from healthy and cancer patients. Through such screening, there would be a high possibility that any difference of transcriptomes between them reflects the transformation process, since both of the specimens are of the same origin.

Furthermore, from the point of view of clinical application, this approach seems to be also desirable. If we can identify bona fide cancer-specific genes from the cells in pancreatic juice, then it becomes realistic to develop a sensitive way to diagnose PDC by reverse-transcription PCR with pancreatic juice which can be obtained with the ERCP procedure.

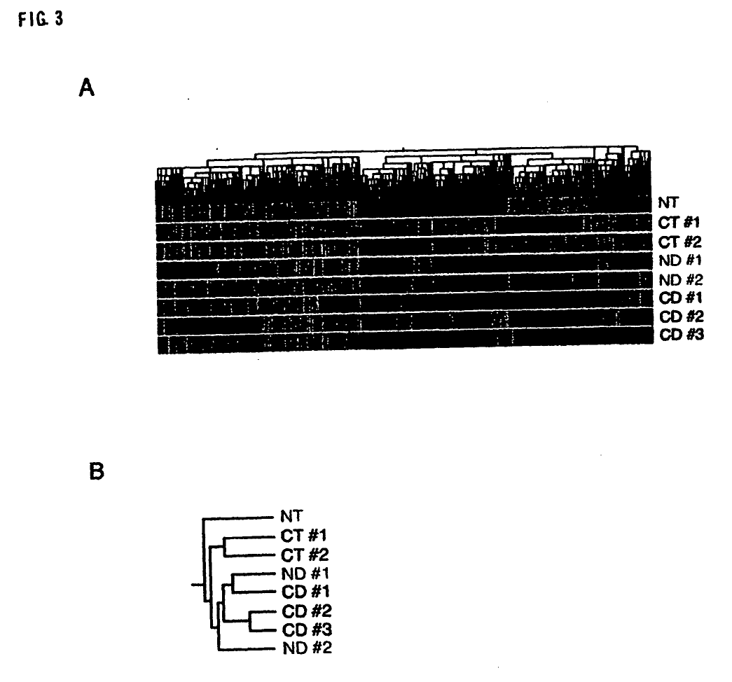

Toward this goal, the expression profiles of 3456 genes were compared among one normal pancreatic tissue, two cancerous pancreatic tissues, two normal ductal cell specimens and three ductal cell specimens obtained from patients with PDC. To visualize the character of transcriptome in each specimen, we conducted a clustering analysis on the data to generate a dendrogram, or a “gene tree”, where genes with similar expression profiles are clustered together (FIG. 3A). From this figure, it is apparent that gene expression pattern of normal ductal cell specimen #1 (ND #1) and those of carcinoma ductal cell specimens (CD #1-3) are similar. Importantly, however, there is yet a significant difference among them, which may include the genes related to the carcinogenesis of pancreas.

To statistically analyze the similarity of transcriptomes in the samples, we then carried out a two-way clustering analysis (Alon, U., Barkai, N., Notterman, D. A., Gish, K., Ybarra, S., Mack, D., and Levine, A. J. Broad patterns of gene expression revealed by clustering analysis of tumor and normal colon tissues probed by oligonucleotide arrays. Proc. Natl. Acad. Sci. USA, 96: 6745-6750, 1999.) to generate a “patient tree” in which specimens with similar transcriptomes are placed nearby. As shown in FIG. 3B, all ductal cell specimens were clustered in the same branch. It was rather surprising to find that transcriptome of cancer ductal cells were more similar to those of normal ductal cells than to those of cancer tissues. The ductal cell specimens from cancer patients #2 and #3 had most similar transcriptomes. The ductal cells of cancer #1 and normal #1 had slightly different, but related, transcriptomes to those of cancer #2 and #3. In contrast, tissue sections from cancer patients (CT #1, 2) were clustered in a separate branch, indicating that transcriptome of resected tissue is very distinct from that of ductal cells.

### EXAMPLE 4

### Potential Molecular Markers for PDC

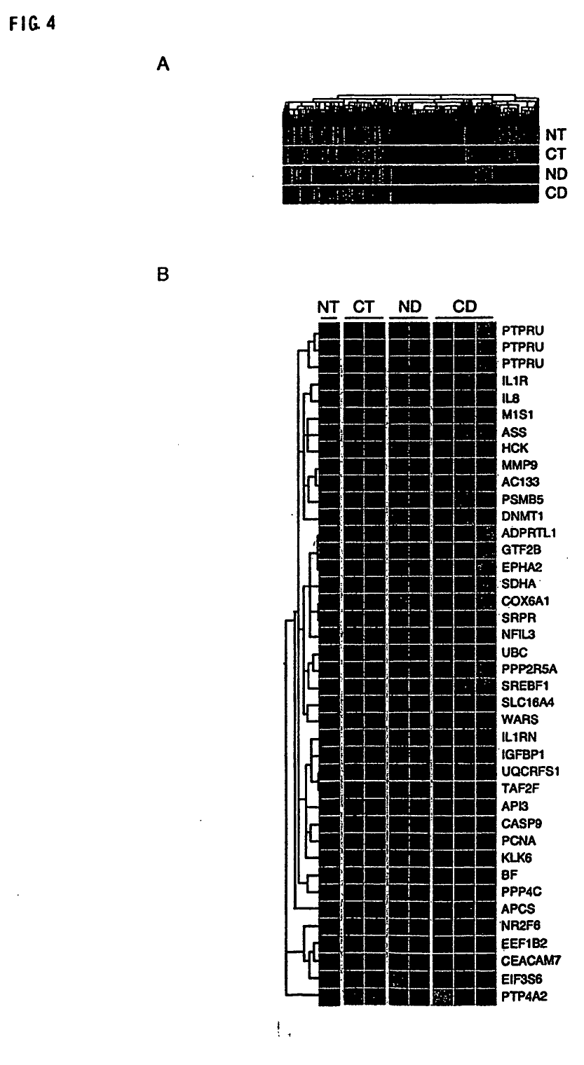

To identify genes that are specifically expressed in the ductal carcinoma cells, the mean expression value of each gene was calculated within every group of cancerous tissue section, ductal cells of healthy individuals and ductal cells of carcinoma patients. Based on these mean values, we then generated another dendrogram, “average tree”, to visualize the clusters of genes whose mean expression was specific to each group (FIG. 4A). In this figure, it is apparent that there are a number of such disease-dependent clusters.

We then tried to extract a set of genes whose expression was induced in the ductal carcinoma cell group, but was negligible or at a very low level in normal tissue- or normal ductal cell-groups. A total of thirty-eight genes were selected, expression of which was kept below 3.0 arbitrary units (U) within normal tissue and normal ductal cell groups, but raised above 15.0 U in, at least, one sample among the cancer ductal cell group (FIG. 4B). Such potential carcinoma-specific molecular markers include the genes for receptor-type protein-tyrosine phosphatase U (PTPRU; GenBank accession No. U73727), membrane component, chromosome1, surface marker 1 (MlSl; X77753), matrix metalloproteinase 9 (MMP9; J05070), AC133 (AF027208), protein phosphatase 2, regulatory subunit B, alpha isoform (PPP2R5A; L42373), Properdin factor B (BF; L15702), amyloid P component, serum (APCS; X04608) and CEACAM7 (X98311). Expression profiles of such genes are shown in FIG. 4B. Interestingly, the expression level of these genes among the cancer tissue samples was weak or negligible (see the “CT” columns), further supporting the superiority of ductal cell-based assay.

The gene names and accession numbers as well as expression intensity data for the genes shown in FIG. 4B are available as Supplementary Information through the website of Cancer Research.

### EXAMPLE 5

### Quantification of mRNA for Potential Pancreatic Ductal Carcinoma-Markers

We then confirmed the gene expression profile by using a “real-time” PCR method. Unamplified cDNAs were prepared from the MUC1⁺ ductal cells obtained from 8 normal individuals and 10 patients with pancreatic carcinoma. A part of O-actin, AC133 or CEACAM7 cDNA was amplified by PCR, and the quantity of the PCR product was monitored in real time, leading to the determination of C_{T} value for each cDNA.

Specifically, first, portions of the unamplified cDNAs were subjected to PCR with SYBR Green PCR Core Reagents (PE Applied Biosystems, Foster City, Calif.). The incorporation of the SYBR Green dye into the PCR products was monitored in real time with an ABI PRISM 7700 sequence detection system (PE Applied Biosystems), thereby allowing determination of the threshold cycle (C_{T}) at which exponential amplification of PCR products begins. The CT values for cDNAs corresponding to the β-actin gene and target genes were used to calculate the abundance of the target transcripts relative to that of β-actin mRNA. The oligonucleotide primers for PCR were as follows: 5′-CCATCATGAAGTGTGACGTGG-3′ (SEQ ID NO: 1) and 5′-GTCCGCCTAGAAGCATTTGCG-3′ (SEQ ID NO: 2) for β-actin cDNA, 5′-CCATCATGAAGTGTGACGTGG-3′ (SEQ ID NO: 3) and 5′-GTCCGCCTAGAAGCATTTGCG-3′ (SEQ ID NO: 4) for carcinoembryonic antigen-related cell adhesion molecule (CEACAM) 7 cDNA, 5′-GAGACTCAGAACACAACCTACCTG-3′ (SEQ ID NO: 5) and 5′-AGCCAGTACTCCAATCATGATGCT-3′ (SEQ ID NO: 6) for AC133 cDNA.

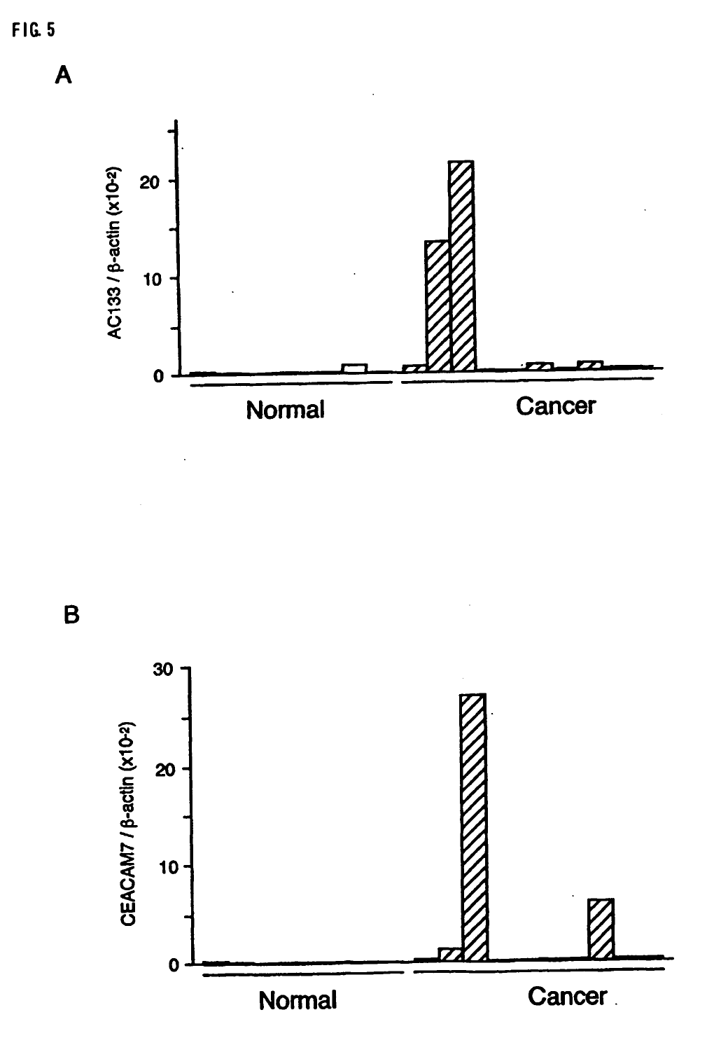

As evident from FIG. 5, in a good agreement with the array data, expression of both AC133 and CEACAM7 genes was highly specific to PDC. Transcription of both genes was almost silent in the normal ductal cells. Therefore, these genes would be the good candidates for PDC-specific markers. It may not be surprising to find that the expression level of AC133 or CEACAM7 gene was diverse even within the cancer specimens, since transformation process of pancreatic ductal cells should not be uniform.

### EXAMPLE 6

### Reassessment of Potential PDC Marker

The expression profiles of 3456 genes were compared among one normal pancreatic tissue, two cancerous pancreatic tissues, three normal pancreatic ductal epitheliums, and six cancerous pancreatic ductal epitheliums similarly to Example 3.

The extraction of the pancreatic ductal carcinoma-specific gene was carried out using the following two standards.

(1) Gene Having Statistically Significant Difference in Expression

First, a gene having significant difference in the gene expression between “normal pancreatic ductal epithelium” and “cancerous pancreatic ductal epithelium” was investigated. The difference in the mean values of the expression in both groups of the respective genes was examined. For this objective, genes were extracted, which genes meet two points: (i) there is a significant difference by P<0.05 in, the mean value using Welch-t-test, and (ii) the expression quantity which was normalized by at least two samples among total nine cases of normal pancreatic ductal epithelium and cancerous pancreatic ductal epithelium exceeds 3. The genes and expression data obtained are shown in Table 1.

TABLE 1
PCa-new 020930

| ID ( )* | 1-NT | 2-CT1 | 2-CT2 | 3-NE1 | 3-NE2 | 3-NE3 | 4-CE1 |
|---|---|---|---|---|---|---|---|
| disease ( )* | 1-Normal | 2-Cancer | 2-Cancer | 1-Normal | 1-Normal | 1-Normal | 2-Cancer |
| sample ( )* | 1-Tissue | 1-Tissue | 1-Tissue | 2-ERCP | 2-ERCP | 2-ERCP | 2-ERCP |
| Class ( )*S | 1-NT | 2-CT | 2-CT | 3-NE | 3-NE | 3-NE | 4-CE |
| Systematic | Normalized | Normalized | Normalized | Normalized | Normalized | Normalized | Normalized |
| 1-1-12-17-B | −1.0437212 | 0.5749708 | −0.9441189 | 2.718987 | 0.8410943 | 1.6470765 | 0.19631301 |
| 1-2-7-3-B | −0.96276224 | −0.9331898 | 0.6457677 | 1.1063426 | −1.4874439 | −0.372387 | 12.864946 |
| 1-2-12-11-C | 1.2590567 | −0.0769462 | 0.4031497 | −0.5472792 | 0.5723457 | 1.7173709 | 1.751451 |
| 2-1-4-3-B | −1.2804605 | −0.5176038 | 1.7882683 | 1.2774487 | −0.7130913 | 6.0706506 | −0.9209023 |
| 2-1-13-4-A | 3.8330538 | 0.9573495 | 1.3901579 | 0.3606252 | 1.0563471 | 5.313286 | −0.0560046 |
| 2-1-1-5-A | 0.056807645 | −0.4493783 | 3.0408463 | 2.5881424 | −1.0033274 | −0.390178 | −0.1377438 |
| 1-2-15-11-B | 0.7078568 | 0.6675541 | 0.4368048 | 0.76963675 | −0.1378399 | 0.26771346 | 2.322482 |

| ID ( )* | 4-CE2 | 4-CE3 | 4-CE4 | 4-CE5 | 4-CE6 | | | |
|---|---|---|---|---|---|---|---|---|
| disease ( )* | 2-Cancer | 2-Cancer | 2-Cancer | 2-Cancer | 2-Cancer | | | |
| sample ( )* | 2-ERCP | 2-ERCP | 2-ERCP | 2-ERCP | 2-ERCP | | | |
| Class ( )*S | 4-CE | 4-CE | 4-CE | 4-CE | 4-CE | | | |
| Systematic | Normalized | Normalized | Normalized | Normalized | Normalized | Common | Genbank | Map |
| 1-1-12-17-B | 10.5567465 | 11.453513 | 1.9542751 | 16.565348 | 8.481729 | UBL1 | U61397 | |
| 1-2-7-3-B | 7.813884 | 6.052212 | −0.1645735 | 2.565377 | 0.81003493 | CSTA | X05978 | |
| 1-2-12-11-C | 6.535972 | 3.324204 | 0.6762424 | 4.149957 | 2.8719711 | ID2 | M97796 | |
| 2-1-4-3-B | 21.000277 | 31.051893 | 1.6738739 | 29.171978 | 23.07758 | NR2F6 | X12794 | |
| 2-1-13-4-A | 24.047033 | 3.8701394 | 22.237062 | 30.643848 | 15.684588 | DUSP11 | AF023917 | |
| 2-1-1-5-A | 4.3491054 | 11.119528 | 9.799812 | 8.215349 | 11.889729 | CDKN1C | U22398 | |
| 1-2-15-11-B | 2.7362695 | 3.4298747 | 2.4548209 | 3.3683946 | 5.129958 | IL17R | U58917 | |

(2) Gene Not Expressed in Normality and Highly Expressed in Either of Carcinomas

A gene having a small standard deviation of expression level in respective groups is apt to be selected in the method (1). Therefore, the gene was selected based on criteria that “a gene is not expressed in normality at all, and highly expressed in either of carcinomas” even if dispersion is great. Namely, genes were selected which meets two points: (i) the expression quantity which was normalized by all points of total four cases of normal pancreatic tissue and normal pancreatic ductal epithelium is less than 1, and (ii) the expression quantity which was normalized by at least one point among six cases of cancerous pancreatic ductal epitheliums is 10 or more. The genes and expression data obtained are shown in Table 2.

TABLE 2
PCa-new 020930

| ID ( )* | 1-NT | 2CT1 | 2-CT2 | 3-NE1 | 3-NE2 | 3-NE3 | 4-CE1 | 4-CE2 |
|---|---|---|---|---|---|---|---|---|
| disease ( )* | 1-Normal | 2-Cancer | 2-Cancer | 1-Normal | 1-Normal | 1-Normal | 2-Cancer | 2-Cancer |
| sample ( )* | 1-Tissue | 1-Tissue | 1-Tissue | 2-ERCP | 2-ERCP | 2-ERCP | 2-ERCP | 2-ERCP |
| Class ( )*S | 1-NT | 2-CT | 2-CT | 3-NE | 3-NE | 3-NE | 4-CE | 4-CE |
| Systematic | Normalized | Normalized | Normalized | Normalized | Normalized | Normalized | Normalized | Normalized |
| 1-2-15-2-C | 0.83086836 | −0.7451082 | 0.3645208 | −0.39570546 | 0.34943935 | 0.22075643 | 0.101790994 | 0.53580064 |
| 1-2-8-10-A | −0.739758 | −1.1833804 | −1.0897253 | 0.57451195 | −1.1196457 | 0.94765204 | −1.2429686 | 10.198083 |
| 2-2-3-1-A | −0.5044281 | −0.9116557 | −0.6719626 | −0.48936418 | −1.0983704 | −0.47953585 | −1.2440282 | 81.91901 |
| 1-2-16-1-A | −0.68407035 | 3.8703 | 1.872287 | −0.42162013 | −1.078422 | −0.021526057 | −1.4333261 | 2.7331777 |
| 2-2-1-8-A | −0.56827796 | −0.64698654 | −0.71393853 | −0.3874762 | −1.1364465 | −0.33753824 | −1.2972667 | 14.434119 |
| 2-1-10-14-A | −0.6432633 | −0.9315149 | −0.70332575 | 0.07820974 | −1.1695235 | 0.4453677 | −1.4406478 | 2.3042953 |
| 2-2-9-3-A | −0.26429713 | −0.69620484 | −0.5887835 | −0.019500472 | −1.1916829 | −0.33006078 | −1.1521485 | −0.38558218 |
| 1-1-7-12-C | −0.15073651 | −1.1999204 | −0.56216216 | −0.86162287 | −0.40339166 | 0.55358666 | −0.62051153 | 11.700114 |
| 2-2-5-3-C | 0.101369835 | −1.1671916 | 0.14146994 | −0.40175503 | −0.60739803 | 0.21933182 | −0.30781502 | 1.2044382 |
| 1-1-7-5-C | −0.90143365 | −0.6783066 | −0.84632033 | 0.8498966 | −1.1259781 | 0.47269246 | −1.1123316 | 32.74072 |
| 1-1-8-3-C | 0.27190134 | −1.2284112 | −0.5277182 | −0.8614135 | 0.14081343 | −0.07426509 | −0.571155 | 0.20583348 |
| 1-2-7-6-C | −0.7041156 | −1.3657701 | −0.36892217 | −0.40374562 | −0.23058599 | 0.52934754 | 2.1582983 | 4.027193 |
| 1-1-2-13-C | −0.8213529 | −1.5194712 | −0.027863069 | 0.025155153 | −0.9324734 | 0.68576384 | −0.9553028 | 7.1569686 |
| 1-2-14-2-C | −0.87909883 | −0.9299503 | −1.0147946 | −0.3071892 | −1.0587149 | −0.29775038 | −0.9558244 | 0.86462784 |
| 2-2-2-17-C | −0.4552567 | −0.28026086 | −0.4334943 | 0.5268296 | −0.5880176 | 0.94869745 | −0.6525445 | 1.6394105 |
| 1-2-3-10-C | −0.70437515 | −1.2810289 | −0.4782932 | −1.1022695 | −0.6427372 | 0.09129483 | −0.65333164 | 2.807904 |
| 1-1-10-15-C | −0.76117915 | −1.2385633 | −0.6183661 | −0.7175303 | −1.0908406 | −0.15466219 | −1.0609658 | 5.4239535 |
| 1-2-8-3-C | −0.8178896 | −0.96559715 | −0.6247408 | −0.7654655 | −1.1152606 | 0.101638705 | −0.9462089 | 3.6533527 |
| 2-2-13-8-C | −0.7486847 | −1.1857016 | 0.70384705 | 0.8728553 | −1.2570522 | 0.58521736 | −1.1342179 | 2.7801363 |
| 1-2-9-11-C | 0.4672931 | −0.5955771 | 0.30751652 | 0.7344966 | −0.9126638 | 0.33627024 | −0.85530245 | 5.089726 |
| 1-2-5-14-C | −0.5736765 | −0.6127118 | −0.3305299 | 0.08612346 | −0.30972734 | 0.35022938 | −0.4662406 | 4.685006 |
| 2-2-4-4-B | −0.82414156 | −0.98518926 | −0.60373944 | 0.32242662 | −1.1228969 | 0.21657208 | −1.2110748 | 9.23975 |
| 2-2-8-13-B | −1.6869609 | −1.0881103 | −0.91945624 | −0.5031823 | −1.4763868 | −0.52089834 | −1.3627844 | −0.2581099 |
| 2-2-13-8-B | −1.3604949 | −1.0632969 | −0.39033806 | −0.24766307 | −1.412114 | −0.36889$$44 | −1.3469836 | 17.8871 |
| 1-1-12-2-B | −0.46669397 | −0.51096165 | −1.8225305 | −0.38654 | −1.0293512 | −0.3999223 | −0.9078021 | 18.004663 |
| 2-2-1-14-B | −1.9896322 | −1.006069 | −0.43987486 | −0.3746848 | −1.253515 | −0.38825566 | −1.0513543 | 5.5376973 |
| 1-1-9-14-B | −3.476159 | −1.0816641 | −0.9852842 | 0.10535935 | −0.8353148 | −0.5234256 | −1.2776353 | −0.24393992 |
| 2-1-5-4-B | −1.3106948 | −1.0971371 | −0.38562328 | −0.21642892 | −1.491443 | 0.23197094 | −1.342949 | 5.7346845 |
| 1-2-8-6-B | −0.9356463 | −0.47715354 | −0.572861 | 0.099833384 | −1.2044939 | −0.38576728 | −1.2073278 | 18.230345 |
| 2-2-12-8-B | −0.016367186 | −0.23514774 | 0.8766775 | 0.11947976 | −1.0497247 | 0.049435224 | −0.8891539 | 2.7569396 |
| 1-2-1-13-B | −0.74208367 | −1.0005603 | −0.67414904 | 0.071623676 | −1.5274999 | −0.26851898 | −1.398162 | 20.148016 |
| 2-2-5-6-B | −0.67134106 | −1.0531477 | −0.45721814 | −0.35351625 | −1.1759236 | −0.32282257 | −1.0548271 | 1.1636503 |
| 2-1-1-3-B | −4.072447 | −1.0601578 | −3.4112713 | −0.121931195 | −1.4412998 | −0.3785295 | −1.3237727 | 27.088139 |
| 2-2-7-8-B | 0.16435957 | 0.26746523 | −0.09567317 | −0.09291312 | 0.34315053 | −0.021658242 | −0.14385706 | −0.01174197 |
| 2-1-1-16-B | −0.06525426 | −0.8277964 | −0.7360943 | −0.24819888 | −1.03572 | −0.08702235 | −0.9003987 | 10.641637 |
| 2-2-10-13-B | −1.8264197 | −1.0761682 | −1.2314644 | −0.47793582 | −1.472417 | −0.50521886 | −1.2833109 | −0.10388584 |
| 2-2-2-3-B | −0.19309351 | −0.49642047 | 1.2532928 | 0.9319025 | 0.011316149 | 0.108761504 | 0.2817736 | 6.633832 |
| 2-1-2-8-B | −1.1684865 | −1.0058678 | −0.6721565 | −0.20009111 | −1.5247263 | 0.9606199 | −1.4011999 | 13.189106 |
| 2-2-6-17-B | −1.4258707 | −0.91643924 | −0.6752391 | 0.23361342 | −1.2002254 | 0.043066103 | −1.2640374 | 5.3032966 |
| 2-1-6-7-B | −1.6499499 | −1.0705028 | −0.578745 | −0.35899436 | −1.4109768 | −0.27476987 | −1.3426595 | 12.775441 |
| 1-1-3-8-B | −1.9702139 | −0.910232 | −0.98260784 | 0.048173465 | −0.92121214 | −0.4621139 | −1.2181952 | 15.75814 |
| 2-2-1-4-B | −2.6147308 | −1.0700614 | −2.4830244 | 0.92508197 | −1.3611406 | 0.51439667 | −1.3110853 | 9.422945 |
| 2-1-1-13-B | −0.70345557 | −0.945123 | 0.28812435 | 0.5858669 | −1.4354106 | 0.093148984 | −1.2376316 | 1.8588752 |
| 2-2-2-9-B | 0.026287146 | −0.9800069 | −0.39045346 | −0.8416284 | −1.3146098 | −0.46645373 | −1.093252 | 0.8277583 |

| ID ( )* | 4-CE3 | 4-CE4 | 4-CE5 | 4-CE6 | | | |
|---|---|---|---|---|---|---|---|
| disease ( )* | 2-Cancer | 2-Cancer | 2-Cancer | 2-Cancer | | | |
| sample ( )* | 2-ERCP | 2-ERCP | 2-ERCP | 2-ERCP | | | |
| Class ( )*S | 4-CE | 4-CE | 4-CE | 4-CE | | | |
| Systematic | Normalized | Normalized | Normalized | Normalized | Common | Genbank | Map |
| 1-2-15-2-C | −0.034824237 | −0.75755817 | 10.234757 | 3.2788074 | HSP105B | AB003334 | |
| 1-2-8-10-A | 0.50131196 | 0.6153933 | −0.24645965 | −0.10642163 | CSF3R | M59818 | |
| 2-2-3-1-A | −0.82954305 | −0.7728402 | 52.955162 | 8.695954 | IGFBP1 | Y00856 | |
| 1-2-16-1-A | 0.10015208 | 18.15875 | 6.8672757 | 1.2175622 | PTP4A2 | L48722 | |
| 2-2-1-8-A | −0.11893914 | −0.6916848 | 1.8572503 | −0.11350694 | ILB | M17017 | |
| 2-1-10-14-A | −0.25959742 | 1.0378929 | 12.197743 | 0.35553208 | TRC8 | AF064801 | |
| 2-2-9-3-A | −0.6656971 | −1.042183 | −0.22644566 | 19.032608 | HTR1B | M81590 | |
| 1-1-7-12-C | 0.8354296 | 1.3147045 | 22.58007 | 1.1595023 | SNRPC | M18465 | |
| 2-2-5-3-C | 0.18429112 | −0.24895646 | 10.236346 | 0.7244216 | BTF3L3 | M90356 | |
| 1-1-7-5-C | 0.34541568 | 4.7400346 | 34.678337 | 24.004997 | GTF2B | M76766 | |
| 1-1-8-3-C | −0.10501541 | −0.24204558 | 25.885078 | 0.5710773 | IRF4 | U52682 | |
| 1-2-7-6-C | 0.33610433 | 0.6720941 | 20.261782 | 3.3251421 | GTF2F2 | X16901 | |
| 1-1-2-13-C | 1.0340236 | −0.13567235 | 10.465545 | 0.31571206 | NDUFB10 | AF088991 | |
| 1-2-14-2-C | −0.50729436 | −0.7682098 | 14.985777 | 2.823051 | GALNT1 | U41514 | |
| 2-2-2-17-C | −0.006969276 | 2.0505915 | 10.673549 | 2.394989 | GTF3C2 | AF054988 | |
| 1-2-3-10-C | −0.111308716 | −0.6392184 | 47.49218 | 1.1911334 | POU2AF1 | Z49194 | |
| 1-1-10-15-C | −0.6149011 | −0.5270072 | 18.120232 | 4.944318 | PON2 | AF001601 | |
| 1-2-8-3-C | −0.1806792 | −0.55605286 | 11.01731 | 0.10982381 | JUN | J04111 | |
| 2-2-13-8-C | −0.39301956 | −0.21640126 | 5.640998 | 11.079223 | PDHB | M34479 | |
| 1-2-9-11-C | 0.36643705 | −0.22752139 | 21.813118 | 0.4961 | SLC16A3 | U81800 | |
| 1-2-5-14-C | 0.04127722 | −0.82435304 | 14.570574 | 7.525957 | HNRPA1 | X06747 | |
| 2-2-4-4-B | 7.2231693 | −0.4544621 | 13.22554 | −0.061598737 | ETS2 | J04102 | |
| 2-2-8-13-B | −0.3379202 | −0.7712904 | −0.3585155 | 22.518734 | F7 | M13232 | |
| 2-2-13-8-B | 2.585355 | −0.75497735 | 2.1687999 | −1.5289917 | IL8 | M17017 | |
| 1-1-12-2-B | −0.013620046 | −0.45548657 | 0.33424222 | −0.070414804 | API3 | U45880 | |
| 2-2-1-14-B | 0.120558 | −0.17630841 | 11.258256 | 0.7329485 | CEACAM5 | M29540 | |
| 1-1-9-14-B | −0.36471263 | −0.70452905 | −0.32896903 | 37.836742 | APOA4 | X13629 | |
| 2-1-5-4-B | 2.3924322 | −0.589106 | 12.725108 | 1.0163765 | ANXA5 | M18366 | |
| 1-2-8-6-B | 6.44725 | −0.6323603 | −0.13214763 | −0.42575398 | MMP9 | J05070 | |
| 2-2-12-8-B | 2.5784667 | −0.31160945 | 12.340426 | 0.843506 | UBE3A | U84404 | |
| 1-2-1-13-B | 8.322666 | −0.4604686 | 0.55440766 | −0.3645801 | AC133 | AF027208 | |
| 2-2-5-6-B | 0.10018486 | −0.59586346 | 12.221879 | −0.1587815 | ADAM9 | U41766 | |
| 2-1-1-3-B | 0.12429178 | −0.7155742 | 61.09496 | 3.6081612 | KLK6 | AF013988 | |
| 2-2-7-8-B | −0.017013976 | −0.120177805 | −0.035136256 | 12.68807 | SCYA3 | M23452 | |
| 2-1-1-16-B | 22.55869 | 0.02142055 | −0.24739353 | −0.011327783 | CEACAM7 | X98311 | |
| 2-2-10-13-B | −0.31294376 | −0.7723413 | −0.35982263 | 522.4977 | MYBPC3 | X84075 | |
| 2-2-2-3-B | 1.7828851 | 0.18269528 | 17.963537 | 2.2447588 | PSMD11 | AB003102 | |
| 2-1-2-8-B | 10.715758 | −0.53639877 | 4.029168 | −0.014978451 | RGS2 | L13391 | |
| 2-2-6-17-B | 0.7861668 | −0.36402935 | 14.707854 | 12.882116 | KCNK1 | U33632 | |
| 2-1-6-7-B | 5.89186 | −0.50547165 | 2.7240427 | 1.213881 | SOD2 | M36693 | |
| 1-1-3-8-B | 0.39688125 | −0.42575938 | 51.523834 | 0.07250065 | ECM1 | U68186 | |
| 2-2-1-4-B | 8.953039 | −0.46971676 | 12.043333 | 1.1761023 | PSMA2 | D00760 | |
| 2-1-1-13-B | 1.8388108 | −0.4318526 | 22.482006 | 0.4615081 | CAMLG | U18242 | |
| 2-2-2-9-B | 0.14289854 | −0.67567885 | 3.50E−04 | 52.211086 | RGS5 | AB008109 | |

SOD2 (superoxide dismutase 2) in Table 2 is an enzyme which deactivates a free radical ion produced in cells, and called as manganese SOD (Mn SOD). SOD2 plays a role of protecting cells from an excessive oxidization state in cells, and a mouse whose SOD2 gene was destroyed dies within 10 days after birth by myocardiopathy and metabolic acidosis. The fact that SOD2 is highly expressed in pancreatic carcinoma indicates that SOD2 possibly plays a role of protecting pancreatic carcinoma cells which is in an excessive propagation condition, from cell death.

With respect to SOD2, for example, it is reported that expression is induced when tumor necrosis factor a (TNF-α) is added to pancreatic carcinoma cell strain (Oncology Research 9:623-627, 1997).

Accordingly, we paid attention to SOD2, and carried out experiments below.

### EXAMPLE 7

### Quantification of mRNA of SOD2

We confirmed the gene expression of SOD2 similarly to Example 5 using a “real time” PCR method. Unamplified cDNAs were prepared from the MUC1⁺ductal cells obtained from pancreatic ductal carcinoma (28 cases), benign tumor (16 cases), chronic pancreatitis (4 cases), and normal pancreatitis (12 cases). β-Actin and a part of SOD2 cDNA was amplified by PCR, and the amount of PCR product was monitored in real time, leading to the determination of C_{T} value for each cDNA.

Specifically, portions of unamplified cDNAs were subjected to PCR with SYBR Green PCR Core Reagents (PE Applied Biosystems, Foster City, Calif.). The incorporation of SYBR Green dye into the PCR products was monitored in real time with an ABI PRISM 7700 sequence detection system (PE Applied Biosystems), thereby allowing determination of the threshold value (C_{T}) at which exponential proliferation of PCR products begins. The C_{T} values for cDNAs corresponding to the β-actin gene and target genes were used to calculate the abundance of the target transcripts relative to that of β-actin mRNA.

Specifically, as oligonucleotide primers amplifying SOD2, sense primer: 5′-CAGGATCCACTGCAAGGAACAACA-3′ (SEQ ID NO: 7) and anti-sense primer: 5′-CATGTATCTTTCAGTTACATTCTC-3′ (SEQ ID NO: 8) were used. Alternatively, as oligonucleotide primers amplifying β-actin for internal control, sense primer: 5′-CCATCATGAAGTGTGACGTGG-3′ (SEQ ID NO: 9) and anti-sense primer: 5′-GTCCGCCTAGAAGCATTTGCG-3′ (SEQ ID NO: 10) were used. The respective primers were reacted 60 times at a cycle of 15 seconds at 94° C., 30 seconds at 60° C., and one minute at 72° C. to calculate the Ct value.

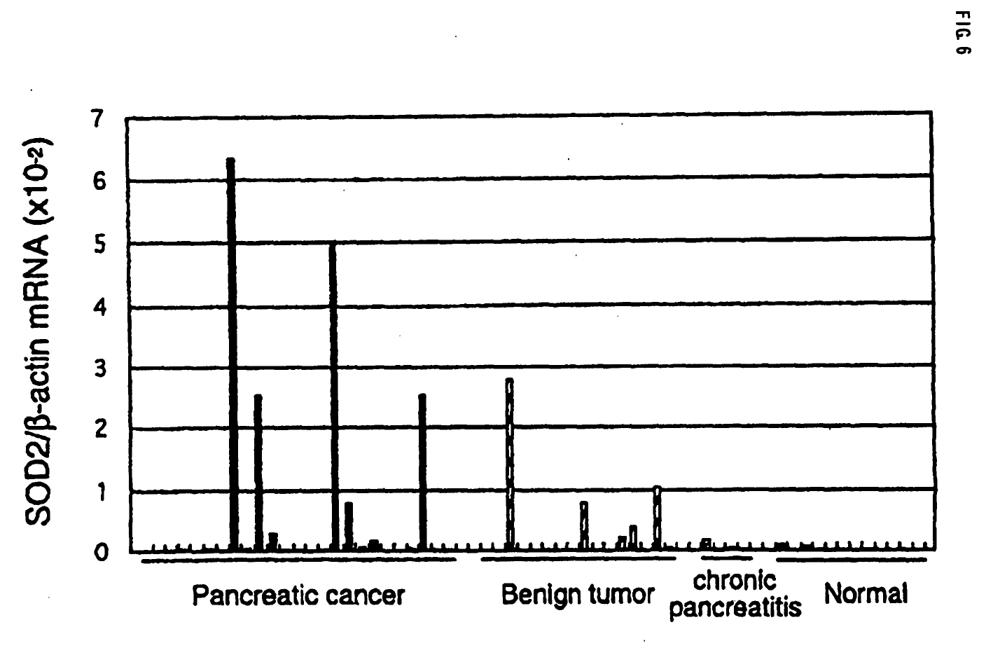

It is apparent from FIG. 6 that SOD2 was detected in about 50% of subjects who were diagnosed as the pancreatic ductal carcinoma patients. The value is very high as the correct diagnosis rate of the pancreatic ductal carcinoma patients.

### EXAMPLE 8

### Quantification of mRNA of CDKN1C, HSP105, IGFBP1, UBE3A, CAPN2 and SOD2

We confirmed the gene expression of CDKN1C, HSP105, IGFBP1, UBE3A, CAPN2 and SOD2 similarly to Example 7 using a “real time” PCR method. The oligonucleotide primers for PCR were as follows: 5′-agagatcagcgcctgagaag-3′ (SEQ ID NO: 11) and 5′-tgggctctaaattggctcac-3′ (SEQ ID NO: 12) for CDKN1C cDNA, 5′-cacagccccaggtacaaact-3′ (SEQ ID NO: 13) and 5′-tttgctttgtcagcatctgg-3′ (SEQ ID NO: 14) for HSP105 cDNA, 5′-ctgccaaactgcaacaagaa-3′ (SEQ ID NO: 15) and 5′-tatctggcagttggggtctc-3′ (SEQ ID NO: 16) for IGFBP1 cDNA, 5′-aagcctgcacgaatgagtt-3′ (SEQ ID NO: 17) and 5′-ggagggatgaggatcacaga-3′ (SEQ ID NO: 18) for UBE3A cDNA, 5′-aggcatacgccaagatcaac-3′ (SEQ ID NO: 19) and 5′-gccaaggagagagccttttt-3′ (SEQ ID NO: 20) for CAPN2 cDNA, 5′-caggatccactgcaaggaacaaca-3′ (SEQ ID NO: 21) and 5′-catgtatctttcagttacattctc-3′ (SEQ ID NO: 22) for SOD2 cDNA, 5′-ccatcatgaagtgtgacgtgg-3′ (SEQ ID NO: 23) and 5′gtccgcctagaagcatttgcg-3′ (SEQ ID NO: 24) for B-actin cDNA.

PCR was conducted to calculate the Ct value. PCR conditions were, 2 minuites at 50° C., 15 minuites at 95° C., and 60 cycles of 15 seconds at 94° C., 30 seconds at 60° C., and one minute at 72° C., in the presence of UNG(Uracil-N-Glycosylase). The expression data obtained are shown in Table 3 to 8 (The abbreviated titles in Tables represent as follows: Ca:pancreatic cancer patients, IPMT: benign tumor patients, Chr.pancreatitis:chronic pancreatitis patients, Normal: normal individuals.)

The ratio of subjects with the value of “2e(act−marker gene)×1000” being more than one to all subjects of each disease is shown in table9, and the ratio of subjects with the value of “2e(act−marker gene)×1000” being more than five to all subjects of each disease is shown in table10.

The ratio of subjects with the value of “2e(act —SOD2)×1000” or “2e(act−HSP105)×1000” being more than one to all subjects of each disease is shown in tables11 and 12.

Further, the ratio of subjects with the value of “2e(act−SOD2)×1000” being more than five or with the value of “2e(act−HSP105)×1000” being more than one to all subjects of each disease is shown in tablesl3 and 14.

TABLE 3

| | |
|---|---|
| Gene | GenBank description |
| SOD2 | M36693 superoxide dismutase 2, mitochondrial |

| | SOD2 | actin | act − SOD | 2e(act − SOD) | 2e(act − SOD) × 1000 |
|---|---|---|---|---|---|
| Ca-1 | 30.43 | 17.54 | −12.89 | 0.000131742 | 0.132 |
| Ca-2 | 27.48 | 14.48 | −13 | 0.00012207 | 0.122 |
| Ca-3 | 28.19 | 14.75 | −13.44 | 8.99823E−05 | 0.090 |
| Ca-4 | 36.37 | 26.55 | −9.82 | 0.001106332 | 1.106 |
| Ca-5 | 50 | 33.04 | −16.96 | 7.84389E−06 | 0.008 |
| Ca-6 | 50 | 32.27 | −17.73 | 4.59979E−06 | 0.005 |
| Ca-7 | 50 | 35.89 | −14.11 | 5.65544E−05 | 0.057 |
| Ca-9 | 50 | 34.09 | −15.91 | 1.6241E−05 | 0.016 |
| Ca-10 | 31.94 | 19.27 | −12.67 | 0.000153444 | 0.153 |
| Ca-11 | 50 | 38.8 | −11.2 | 0.000425074 | 0.425 |
| Ca-12 | 50 | 25.44 | −24.56 | 4.043E−08 | 0.000 |
| Ca-13 | 50 | 38.23 | −11.77 | 0.000286337 | 0.286 |
| Ca-14 | 39.54 | 26.36 | −13.18 | 0.000107752 | 0.108 |
| Ca-16 | 39.33 | 35.35 | −3.98 | 0.063372467 | 63.372 |
| Ca-17 | 41.34 | 30.03 | −11.31 | 0.000393868 | 0.394 |
| Ca-18 | 39.82 | 34.51 | −5.31 | 0.025207555 | 25.208 |
| Ca-20 | 38.18 | 29.79 | −8.39 | 0.002980975 | 2.981 |
| Ca-21 | 43.07 | 30.19 | −12.88 | 0.000132658 | 0.133 |
| Ca-22 | 41.59 | 29.06 | −12.53 | 0.000169081 | 0.169 |
| Ca-24 | 50 | 33.4 | −16.6 | 1.0067E−05 | 0.010 |
| Ca-25 | 50 | 37.78 | −12.22 | 0.000209611 | 0.210 |
| Ca-26 | 42.11 | 37.79 | −4.32 | 0.050066867 | 50.067 |
| Ca-27 | 42.19 | 35.16 | −7.03 | 0.007651721 | 7.652 |
| Ca-28 | 50 | 39.07 | −10.93 | 0.000512557 | 0.513 |
| Ca-29 | 32.37 | 23.2 | −9.17 | 0.001736021 | 1.736 |
| Ca-30 | 60 | 33.76 | −26.24 | 1.26175E−08 | 0.000 |
| Ca-31 | 60 | 38.41 | −21.59 | 3.16783E−07 | 0.000 |
| Ca-32 | 38.28 | 27.09 | −11.19 | 0.00042803 | 0.428 |
| Ca-33 | 34.46 | 29.15 | −5.31 | 0.025207555 | 25.208 |
| Ca-34 | 38.2 | 25.92 | −12.28 | 0.000201072 | 0.201 |
| Ca-35 | 39.72 | 26.89 | −12.83 | 0.000137336 | 0.137 |
| Ca-36 | 40.73 | 28.24 | −12.49 | 0.000173834 | 0.174 |
| Ca-37 | 35.45 | 21.29 | −14.16 | 5.4628E−05 | 0.055 |
| Ca-38 | 23.86 | 17.23 | −6.63 | 0.010096506 | 10.097 |
| Ca-39 | 60 | 34.68 | −25.32 | 2.38737E−08 | 0.000 |
| IPMT-1 | 50 | 35.53 | −14.47 | 4.40652E−05 | 0.044 |
| IPMT-2 | 50 | 36.05 | −13.95 | 6.31876E−05 | 0.063 |
| IPMT-4 | 50 | 36.04 | −13.96 | 6.27511E−05 | 0.063 |
| IPMT-5 | 50 | 34.1 | −15.9 | 1.6354E−05 | 0.016 |
| IPMT-6 | 50 | 38.11 | −11.89 | 0.000263483 | 0.263 |
| IPMT-7 | 50 | 35.14 | −14.86 | 3.36275E−05 | 0.034 |
| IPMT-8 | 41.4 | 35.01 | −6.39 | 0.0119239 | 11.924 |
| IPMT-9 | 50 | 34.07 | −15.93 | 1.60174E−05 | 0.016 |
| IPMT-11 | 40.8 | 33.77 | −7.03 | 0.007651721 | 7.652 |
| IPMT-12 | 50 | 35.13 | −14.87 | 3.33952E−05 | 0.033 |
| IPMT-13 | 50 | 33.57 | −16.43 | 1.1326E−05 | 0.011 |
| IPMT-14 | 40.83 | 31.98 | −8.85 | 0.002167128 | 2.167 |
| IPMT-15 | 60 | 34.36 | −25.64 | 1.91245E−08 | 0.000 |
| IPMT-16 | 39.24 | 32.63 | −6.61 | 0.010237448 | 10.237 |
| IPMT-17 | 38.23 | 27.1 | −11.13 | 0.000446207 | 0.446 |
| IPMT-18 | 36.85 | 24.06 | −12.79 | 0.000141197 | 0.141 |
| IPMT-19 | 42.2 | 30.2 | −12 | 0.000244141 | 0.244 |
| chr. pancreatitis-1 | 60 | 35.13 | −24.87 | 3.26125E−08 | 0.000 |
| chr. pancreatitis-2 | 60 | 34.39 | −25.61 | 1.95264E−08 | 0.000 |
| chr. pancreatitis-3 | 41.55 | 29.32 | −12.23 | 0.000208163 | 0.208 |
| chr. pancreatitis-4 | 43.52 | 34.26 | −9.26 | 0.001631031 | 1.631 |
| chr. pancreatitis-5 | 41.13 | 31.67 | −9.46 | 0.001419895 | 1.420 |
| chr. pancreatitis-6 | 60 | 33.61 | −26.39 | 1.13715E−08 | 0.000 |
| chr. pancreatitis-7 | 41.65 | 33.83 | −7.82 | 0.004425328 | 4.425 |
| chr. pancreatitis-8 | 60 | 35.6 | −24.4 | 4.51719E−08 | 0.000 |
| normal-1 | 40.37 | 30.16 | −10.21 | 0.000844275 | 0.844 |
| normal-2 | 60 | 38.34 | −21.66 | 3.0178E−07 | 0.000 |
| normal-3 | 60 | 34.47 | −25.53 | 2.06397E−08 | 0.000 |
| normal-4 | 60 | 32.96 | −27.04 | 7.24684E−09 | 0.000 |
| normal-5 | 60 | 32.94 | −27.06 | 7.14707E−09 | 0.000 |
| normal-6 | 60 | 30.57 | −29.43 | 1.38257E−09 | 0.000 |
| normal-7 | 50 | 35.63 | −14.37 | 4.72279E−05 | 0.047 |
| normal-8 | 50 | 39.15 | −10.85 | 0.000541782 | 0.542 |
| normal-9 | 50 | 35.34 | −14.66 | 3.86278E−05 | 0.039 |
| normal-10 | 50 | 36.04 | −13.96 | 6.27511E−05 | 0.063 |
| normal-11 | 50 | 35.81 | −14.19 | 5.35038E−05 | 0.054 |
| normal-12 | 50 | 30.72 | −19.28 | 1.57088E−06 | 0.002 |
| normal-13 | 50 | 32.1 | −17.9 | 4.08849E−06 | 0.004 |
| normal-14 | 50 | 31.05 | −18.95 | 1.97461E−06 | 0.002 |
| normal-15 | 50 | 33.22 | −16.78 | 8.88621E−06 | 0.009 |
| normal-16 | 60 | 34.53 | −25.47 | 2.15162E−08 | 0.000 |
| normal-17 | 60 | 28.2 | −31.8 | 2.67452E−10 | 0.000 |
| normal-19 | 60 | 39.15 | −20.85 | 5.29084E−07 | 0.001 |
| normal-20 | 60 | 38.13 | −21.87 | 2.609E−07 | 0.000 |
| normal-21 | 60 | 34.54 | −25.46 | 2.16659E−08 | 0.000 |
| normal-22 | 60 | 36.06 | −23.94 | 6.21358E−08 | 0.000 |
| normal-23 | 60 | 38.23 | −21.77 | 2.79626E−07 | 0.000 |
| normal-26 | 60 | 35.1 | −24.9 | 3.19413E−08 | 0.000 |
| normal-27 | 60 | 38.44 | −21.56 | 3.2344E−07 | 0.000 |
| normal-28 | 60 | 32.28 | −27.72 | 4.52323E−09 | 0.000 |
| normal-29 | 60 | 36.76 | −23.24 | 1.0094E−07 | 0.000 |
| normal-30 | 60 | 33.34 | −26.66 | 9.43062E−09 | 0.000 |

TABLE 4

| | |
|---|---|
| Gene | GenBank description |
| IGFBP1 | Y00856 insulin-like growth factor binding protein 1 |

IGFBP1

| | IGFBP1 | actin | act − IGF | 2e(act − IGF) | 2e(act − IGF) × 1000 |
|---|---|---|---|---|---|
| Ca-1 | 29.09 | 23.19 | −5.9 | 0.01674646 | 16.746 |
| Ca-2 | 60 | 34.14 | −25.86 | 1.64197E−08 | 0.000 |
| Ca-3 | 60 | 49.1 | −10.9 | 0.000523327 | 0.523 |
| Ca-4 | 26.87 | 20.16 | −6.71 | 0.009551877 | 9.552 |
| Ca-5 | 33.66 | 24.65 | −9.01 | 0.001939634 | 1.940 |
| Ca-6 | 60 | 26.82 | −33.18 | 1.0276E−10 | 0.000 |
| Ca-7 | 60 | 32.1 | −27.9 | 3.99267E−09 | 0.000 |
| Ca-8 | 60 | 32.75 | −27.25 | 6.26517E−09 | 0.000 |
| Ca-9 | 60 | 35.85 | −24.15 | 5.37187E−08 | 0.000 |
| Ca-10 | 60 | 32.06 | −27.94 | 3.88349E−09 | 0.000 |
| Ca-11 | 60 | 27.65 | −32.35 | 1.82675E−10 | 0.000 |
| Ca-12 | 60 | 33.06 | −26.94 | 7.76698E−09 | 0.000 |
| Ca-13 | 60 | 36 | −24 | 5.96046E−08 | 0.000 |
| Ca-14 | 60 | 40.05 | −19.95 | 9.87306E−07 | 0.001 |
| Ca-15 | 60 | 32.49 | −27.51 | 5.23196E−09 | 0.000 |
| Ca-16 | 60 | 30.4 | −29.6 | 1.22889E−09 | 0.000 |
| Ca-17 | 60 | 25.4 | −34.6 | 3.84027E−11 | 0.000 |
| Ca-18 | 36.18 | 27.43 | −8.75 | 0.00232267 | 2.323 |
| Ca-19 | 46.64 | 31.85 | −14.79 | 3.52993E−05 | 0.035 |
| Ca-20 | 34.68 | 29.3 | −5.38 | 0.024013675 | 24.014 |
| Ca-21 | 25.25 | 14.87 | −10.38 | 0.000750427 | 0.750 |
| Ca-22 | 35.21 | 22.15 | −13.06 | 0.000117098 | 0.117 |
| Ca-23 | 19.61 | 23.07 | 3.46 | 11.00433455 | 11004.335 |
| IPMT-1 | 60 | 34.52 | −25.48 | 2.13676E−08 | 0.000 |
| IPMT-2 | 60 | 26.76 | −33.24 | 9.85741E−11 | 0.000 |
| IPMT-3 | 60 | 37.43 | −22.57 | 1.60603E−07 | 0.000 |
| IPMT-4 | 60 | 38.2 | −21.8 | 2.73871E−07 | 0.000 |
| IPMT-5 | 60 | 39.2 | −20.8 | 5.47742E−07 | 0.001 |
| IPMT-6 | 60 | 37.2 | −22.8 | 1.36936E−07 | 0.000 |
| IPMT-7 | 60 | 44.49 | −15.51 | 2.14301E−05 | 0.021 |
| IPMT-8 | 60 | 38.78 | −21.22 | 4.09396E−07 | 0.000 |
| IPMT-9 | 60 | 33.59 | −26.41 | 1.1215E−08 | 0.000 |
| IPMT-10 | 60 | 35.05 | −24.95 | 3.08533E−08 | 0.000 |
| IPMT-11 | 60 | 35.08 | −24.92 | 3.15016E−08 | 0.000 |
| IPMT-12 | 60 | 34.44 | −25.56 | 2.0215E−08 | 0.000 |
| IPMT-13 | 60 | 45.41 | −14.59 | 4.05483E−05 | 0.041 |
| IPMT-14 | 60 | 33.64 | −26.36 | 1.16105E−08 | 0.000 |
| IPMT-15 | 60 | 28.59 | −31.41 | 3.50468E−10 | 0.000 |
| IPMT-16 | 60 | 38.32 | −21.68 | 2.97625E−07 | 0.000 |
| IPMT-17 | 60 | 30.27 | −29.73 | 1.123E−09 | 0.000 |
| Chr. pancreatitis-1 | 60 | 30.51 | −29.49 | 1.32625E−09 | 0.000 |
| Chr. pancreatitis-2 | 60 | 38.52 | −21.48 | 3.41882E−07 | 0.000 |
| Chr. pancreatitis-3 | 60 | 47.5 | −12.5 | 0.000172633 | 0.173 |
| Chr. pancreatitis-4 | 60 | 30.6 | −29.4 | 1.41162E−09 | 0.000 |
| Chr. pancreatitis-5 | 60 | 29.4 | −30.6 | 6.14444E−10 | 0.000 |
| Chr. pancreatitis-6 | 60 | 32.2 | −27.8 | 4.27923E−09 | 0.000 |
| Chr. pancreatitis-7 | 60 | 37.88 | −22.12 | 2.1939E−07 | 0.000 |
| Normal-2 | 60 | 30.53 | −29.47 | 1.34476E−09 | 0.000 |
| Normal-3 | 60 | 36.1 | −23.9 | 6.38827E−08 | 0.000 |
| Normal-4 | 60 | 35.1 | −24.9 | 3.19413E−08 | 0.000 |
| Normal-5 | 60 | 36.46 | −23.54 | 8.19887E−08 | 0.000 |
| Normal-6 | 60 | 34.3 | −25.7 | 1.83455E−08 | 0.000 |
| Normal-7 | 60 | 34.64 | −25.36 | 2.32209E−08 | 0.000 |
| Normal-8 | 60 | 45.34 | −14.66 | 3.86278E−05 | 0.039 |
| Normal-9 | 60 | 41.09 | −18.91 | 2.03013E−06 | 0.002 |
| Normal-10 | 60 | 33.57 | −26.43 | 1.10606E−08 | 0.000 |
| Normal-11 | 60 | 35.7 | −24.3 | 4.8414E−08 | 0.000 |
| Normal-12 | 60 | 33.2 | −26.8 | 8.55847E−09 | 0.000 |
| Normal-13 | 60 | 39.25 | −20.75 | 5.67058E−07 | 0.001 |
| Normal-14 | 60 | 31.31 | −28.69 | 2.30914E−09 | 0.000 |
| Normal-15 | 60 | 27.52 | −32.48 | 1.66934E−10 | 0.000 |
| Normal-16 | 60 | 29.54 | −30.46 | 6.77059E−10 | 0.000 |
| Normal-17 | 60 | 35.41 | −24.59 | 3.95979E−08 | 0.000 |
| Normal-18 | 60 | 44.25 | −15.75 | 1.81459E−05 | 0.018 |
| Normal-19 | 60 | 34.04 | −25.96 | 1.53201E−08 | 0.000 |
| Normal-20 | 60 | 33.21 | −26.79 | 8.618E−09 | 0.000 |
| Normal-22 | 60 | 36.2 | −23.8 | 6.84678E−08 | 0.000 |
| Normal-23 | 60 | 30.55 | −29.45 | 1.36354E−09 | 0.000 |
| Normal-24 | 60 | 33.52 | −26.48 | 1.06838E−08 | 0.000 |
| Normal-25 | 60 | 34.56 | −25.44 | 2.19683E−08 | 0.000 |

TABLE 5

| | |
|---|---|
| Gene | GenBank description |
| CDKN1C | cyclin-dependent kinase inhibitor 1C (p57, |
| | U22398 Kip2) |

CDKN1C

| | CDKN1C | actin | act − CDK | 2e(act − CDK) | 2e(act − CDK) × 1000 |
|---|---|---|---|---|---|
| Ca-1 | 35.02 | 24.61 | −10.41 | 0.000734984 | 0.735 |
| Ca-2 | 60 | 34.14 | −25.86 | 1.64197E−08 | 0.000 |
| Ca-3 | 60 | 39.26 | −20.74 | 5.71002E−07 | 0.001 |
| Ca-4 | 60 | 49.1 | −10.9 | 0.000523327 | 0.523 |
| Ca-5 | 29.04 | 20.16 | −8.88 | 0.002122529 | 2.123 |
| Ca-6 | 60 | 34.34 | −25.66 | 1.88612E−08 | 0.000 |
| Ca-7 | 34.72 | 25.03 | −9.69 | 0.001210652 | 1.211 |
| Ca-8 | 60 | 32.56 | −27.44 | 5.49208E−09 | 0.000 |
| Ca-9 | 60 | 26.82 | −33.18 | 1.0276E−10 | 0.000 |
| Ca-10 | 60 | 32.1 | −27.9 | 3.99267E−09 | 0.000 |
| Ca-11 | 60 | 32.75 | −27.25 | 6.26517E−09 | 0.000 |
| Ca-12 | 60 | 35.85 | −24.15 | 5.37187E−08 | 0.000 |
| Ca-13 | 60 | 32.44 | −27.56 | 5.05374E−09 | 0.000 |
| Ca-14 | 26.79 | 32.06 | 5.27 | 38.58585049 | 38585.850 |
| Ca-15 | 37.53 | 29.26 | −8.27 | 0.003239529 | 3.240 |
| Ca-16 | 60 | 33.06 | −26.94 | 7.76698E−09 | 0.000 |
| Ca-17 | 60 | 36 | −24 | 5.96046E−08 | 0.000 |
| Ca-18 | 60 | 39.23 | −20.77 | 5.59251E−07 | 0.001 |
| Ca-19 | 60 | 35.3 | −24.7 | 3.6691E−08 | 0.000 |
| Ca-20 | 60 | 34.74 | −25.26 | 2.48876E−08 | 0.000 |
| Ca-21 | 37.11 | 28.32 | −8.79 | 0.002259157 | 2.259 |
| Ca-22 | 37.15 | 27.35 | −9.8 | 0.001121776 | 1.122 |
| Ca-23 | 37.4 | 28.26 | −9.14 | 0.001772498 | 1.772 |
| Ca-24 | 60 | 31.85 | −28.15 | 3.35742E−09 | 0.000 |
| Ca-25 | 60 | 30.74 | −29.26 | 1.55547E−09 | 0.000 |
| Ca-26 | 31.56 | 21.28 | −10.28 | 0.000804288 | 0.804 |
| Ca-27 | 30.74 | 19.81 | −10.93 | 0.000512557 | 0.513 |
| Ca-28 | 18.84 | 23.07 | 4.23 | 18.76535919 | 18765.359 |
| IPMT-1 | 60 | 34.52 | −25.48 | 2.13676E−08 | 0.000 |
| IPMT-2 | 60 | 26.76 | −33.24 | 9.85741E−11 | 0.000 |
| IPMT-3 | 60 | 37.43 | −22.57 | 1.60603E−07 | 0.000 |
| IPMT-4 | 60 | 38.2 | −21.8 | 2.73871E−07 | 0.000 |
| IPMT-5 | 60 | 37.2 | −22.8 | 1.36936E−07 | 0.000 |
| IPMT-6 | 60 | 44.49 | −15.51 | 2.14301E−05 | 0.021 |
| IPMT-7 | 60 | 38.78 | −21.22 | 4.09396E−07 | 0.000 |
| IPMT-8 | 60 | 33.59 | −26.41 | 1.1215E−08 | 0.000 |
| IPMT-9 | 60 | 35.05 | −24.95 | 3.08533E−08 | 0.000 |
| IPMT-10 | 60 | 34.77 | −25.23 | 2.54105E−08 | 0.000 |
| IPMT-11 | 60 | 34.44 | −25.56 | 2.0215E−08 | 0.000 |
| IPMT-12 | 60 | 33.11 | −26.89 | 8.04088E−09 | 0.000 |
| IPMT-13 | 60 | 32.34 | −27.66 | 4.71531E−09 | 0.000 |
| IPMT-14 | 60 | 28.41 | −31.59 | 3.09359E−10 | 0.000 |
| IPMT-15 | 60 | 38.32 | −21.68 | 2.97625E−07 | 0.000 |
| IPMT-16 | 60 | 30.27 | −29.73 | 1.123E−09 | 0.000 |
| chr. pancreatitis-1 | 60 | 38.52 | −21.48 | 3.41882E−07 | 0.000 |
| chr. pancreatitis-2 | 60 | 47.5 | −12.5 | 0.000172633 | 0.173 |
| chr. pancreatitis-3 | 60 | 30.51 | −29.49 | 1.32625E−09 | 0.000 |
| chr. pancreatitis-4 | 60 | 31.54 | −28.46 | 2.70823E−09 | 0.000 |
| chr. pancreatitis-5 | 60 | 33.68 | −26.32 | 1.19369E−08 | 0.000 |
| chr. pancreatitis-6 | 60 | 37.88 | −22.12 | 2.1939E−07 | 0.000 |
| Normal-2 | 60 | 30.53 | −29.47 | 1.34476E−09 | 0.000 |
| Normal-3 | 60 | 36.1 | −23.9 | 6.38827E−08 | 0.000 |
| Normal-4 | 60 | 35.1 | −24.9 | 3.19413E−08 | 0.000 |
| Normal-5 | 60 | 39.2 | −20.8 | 5.47742E−07 | 0.001 |
| Normal-6 | 60 | 36.46 | −23.54 | 8.19887E−08 | 0.000 |
| Normal-7 | 60 | 34.3 | −25.7 | 1.83455E−08 | 0.000 |
| Normal-8 | 60 | 34.64 | −25.36 | 2.32209E−08 | 0.000 |
| Normal-9 | 60 | 45.34 | −14.66 | 3.86278E−05 | 0.039 |
| Normal-10 | 60 | 41.09 | −18.91 | 2.03013E−06 | 0.002 |
| Normal-11 | 60 | 33.57 | −26.43 | 1.10606E−08 | 0.000 |
| Normal-12 | 60 | 35.7 | −24.3 | 4.8414E−08 | 0.000 |
| Normal-13 | 60 | 33.2 | −26.8 | 8.55847E−09 | 0.000 |
| Normal-14 | 60 | 37.95 | −22.05 | 2.30297E−07 | 0.000 |
| Normal-15 | 60 | 31.5 | −28.5 | 2.63418E−09 | 0.000 |
| Normal-16 | 60 | 30.71 | −29.29 | 1.52346E−09 | 0.000 |
| Normal-17 | 60 | 33.2 | −26.8 | 8.55847E−09 | 0.000 |
| Normal-18 | 60 | 37.68 | −22.32 | 1.9099E−07 | 0.000 |
| Normal-19 | 60 | 44.25 | −15.75 | 1.81459E−05 | 0.018 |
| Normal-20 | 60 | 34.04 | −25.96 | 1.53201E−08 | 0.000 |
| Normal-21 | 60 | 46.17 | −13.83 | 6.86681E−05 | 0.069 |
| Normal-22 | 60 | 48.84 | −11.16 | 0.000437024 | 0.437 |
| Normal-23 | 60 | 36.2 | −23.8 | 6.84678E−08 | 0.000 |
| Normal-24 | 60 | 30.55 | −29.45 | 1.36354E−09 | 0.000 |
| Normal-25 | 60 | 33.52 | −26.48 | 1.06838E−08 | 0.000 |
| Normal-26 | 60 | 34.56 | −25.44 | 2.19683E−08 | 0.000 |

TABLE 6

| | |
|---|---|
| Gene | GenBank description |
| UBE3A | U84404 ubiquitin protein ligase E3A |

| | | | act − | 2e(act − | 2e(act − |
|---|---|---|---|---|---|
| | UBE | actin | UBE | UBE) | UBE) × 1000 |
| Ca-1 | 33.25 | 24.58 | −8.67 | 0.002455104 | 2.455 |
| Ca-2 | 60 | 34.14 | −25.86 | 1.64197E−08 | 0.000 |
| Ca-3 | 60 | 39.26 | −20.74 | 5.71002E−07 | 0.001 |
| Ca-4 | 60 | 49.1 | −10.9 | 0.000523327 | 0.523 |
| Ca-5 | 27.9 | 20.16 | −7.74 | 0.004677651 | 4.678 |
| Ca-6 | 60 | 34.34 | −25.66 | 1.88612E−08 | 0.000 |
| Ca-7 | 60 | 27.26 | −32.74 | 1.39405E−10 | 0.000 |
| Ca-8 | 60 | 32.56 | −27.44 | 5.49208E−09 | 0.000 |
| Ca-9 | 36.21 | 26.82 | −9.39 | 0.001490488 | 1.490 |
| Ca-10 | 35.37 | 32.1 | −3.27 | 0.103664943 | 103.665 |
| Ca-11 | 35.9 | 32.75 | −3.15 | 0.112656308 | 112.656 |
| Ca-12 | 60 | 35.85 | −24.15 | 5.37187E−08 | 0.000 |
| Ca-13 | 60 | 29.77 | −30.23 | 7.94078E−10 | 0.000 |
| Ca-14 | 35.07 | 33.06 | −2.01 | 0.248273124 | 248.273 |
| Ca-15 | 60 | 36 | −24 | 5.96046E−08 | 0.000 |
| Ca-16 | 60 | 39.23 | −20.77 | 5.59251E−07 | 0.001 |
| Ca-17 | 60 | 34.6 | −25.4 | 2.25859E−08 | 0.000 |
| Ca-18 | 60 | 28.47 | −31.53 | 3.22496E−10 | 0.000 |
| Ca-19 | 60 | 28.14 | −31.86 | 2.56557E−10 | 0.000 |
| Ca-20 | 35.09 | 27.52 | −7.57 | 0.005262631 | 5.263 |
| Ca-21 | 34.67 | 30.27 | −4.4 | 0.047366143 | 47.366 |
| Ca-22 | 60 | 31.85 | −28.15 | 3.35742E−09 | 0.000 |
| Ca-23 | 60 | 31.93 | −28.07 | 3.54885E−09 | 0.000 |
| Ca-24 | 27.13 | 19.69 | −7.44 | 0.005758864 | 5.759 |
| Ca-25 | 29.79 | 22.08 | −7.71 | 0.004775939 | 4.776 |
| Ca-26 | 30.8 | 23.07 | −7.73 | 0.004710187 | 4.710 |
| IPMT-1 | 60 | 34.52 | −25.48 | 2.13676E−08 | 0.000 |
| IPMT-2 | 60 | 26.76 | −33.24 | 9.85741E−11 | 0.000 |
| IPMT-3 | 60 | 37.43 | −22.57 | 1.60603E−07 | 0.000 |
| IPMT-4 | 60 | 38.2 | −21.8 | 2.73871E−07 | 0.000 |
| IPMT-5 | 60 | 39.2 | −20.8 | 5.47742E−07 | 0.001 |
| IPMT-6 | 60 | 37.2 | −22.8 | 1.36936E−07 | 0.000 |
| IPMT-7 | 60 | 44.49 | −15.51 | 2.14301E−05 | 0.021 |
| IPMT-8 | 60 | 38.78 | −21.22 | 4.09396E−07 | 0.000 |
| IPMT-9 | 57.55 | 33.59 | −23.96 | 6.12804E−08 | 0.000 |
| IPMT-10 | 60 | 35.05 | −24.95 | 3.08533E−08 | 0.000 |
| IPMT-11 | 60 | 33.32 | −26.68 | 9.30079E−09 | 0.000 |
| IPMT-12 | 60 | 30.11 | −29.89 | 1.00511E−09 | 0.000 |
| IPMT-13 | 35.16 | 33.27 | −1.89 | 0.269807059 | 269.807 |
| IPMT-14 | 60 | 33.37 | −26.63 | 9.62878E−09 | 0.000 |
| IPMT-15 | 60 | 27.29 | −32.71 | 1.42334E−10 | 0.000 |
| IPMT-16 | 60 | 38.32 | −21.68 | 2.97625E−07 | 0.000 |
| IPMT-17 | 60 | 30.27 | −29.73 | 1.123E−09 | 0.000 |
| Chr. | 60 | 38.52 | −21.48 | 3.41882E−07 | 0.000 |
| Pancreatitis-1 | | | | | |
| Chr. | 60 | 47.5 | −12.5 | 0.000172633 | 0.173 |
| Pancreatitis-2 | | | | | |
| Chr. | 38.69 | 30.51 | −8.18 | 0.003448059 | 3.448 |
| Pancreatitis-3 | | | | | |
| Chr. | 60 | 30.46 | −29.54 | 1.28107E−09 | 0.000 |
| Pancreatitis-4 | | | | | |
| Chr. | 60 | 31.22 | −28.78 | 2.16949E−09 | 0.000 |
| Pancreatitis-5 | | | | | |
| Chr. | 60 | 36.45 | −23.55 | 8.14223E−08 | 0.000 |
| Pancreatitis-6 | | | | | |
| Chr. | 60 | 37.88 | −22.12 | 2.1939E−07 | 0.000 |
| Pancreatitis-7 | | | | | |
| Normal-1 | 60 | 42.6 | −17.4 | 5.782E−06 | 0.006 |
| Normal-2 | 60 | 34.35 | −25.65 | 1.89924E−08 | 0.000 |
| Normal-3 | 60 | 34.22 | −25.78 | 1.73559E−08 | 0.000 |
| Normal-4 | 60 | 34.56 | −25.44 | 2.19683E−08 | 0.000 |
| Normal-6 | 60 | 30.84 | −29.16 | 1.66711E−09 | 0.000 |
| Normal-7 | 60 | 31.42 | −28.58 | 2.49208E−09 | 0.000 |
| Normal-8 | 60 | 34.44 | −25.56 | 2.0215E−08 | 0.000 |
| Normal-9 | 60 | 35.75 | −24.25 | 5.01213E−08 | 0.000 |
| Normal-10 | 60 | 40.07 | −19.93 | 1.00109E−06 | 0.001 |
| Normal-11 | 58.35 | 37.56 | −20.79 | 5.51552E−07 | 0.001 |
| Normal-12 | 60 | 36.2 | −23.8 | 6.84678E−08 | 0.000 |
| Normal-14 | 60 | 30.53 | −29.47 | 1.34476E−09 | 0.000 |
| Normal-15 | 60 | 36.1 | −23.9 | 6.38827E−08 | 0.000 |
| Normal-16 | 60 | 35.1 | −24.9 | 3.19413E−08 | 0.000 |
| Normal-17 | 60 | 36.46 | −23.54 | 8.19887E−08 | 0.000 |
| Normal-18 | 60 | 34.3 | −25.7 | 1.83455E−08 | 0.000 |
| Normal-19 | 60 | 34.64 | −25.36 | 2.32209E−08 | 0.000 |
| Normal-20 | 60 | 45.34 | −14.66 | 3.86278E−05 | 0.039 |
| Normal-21 | 60 | 32.44 | −27.56 | 5.05374E−09 | 0.000 |
| Normal-22 | 60 | 32.06 | −27.94 | 3.88349E−09 | 0.000 |
| Normal-23 | 60 | 41.09 | −18.91 | 2.03013E−06 | 0.002 |
| Normal-24 | 60 | 33.57 | −26.43 | 1.10606E−08 | 0.000 |
| Normal-25 | 60 | 35.7 | −24.3 | 4.8414E−08 | 0.000 |
| Normal-26 | 60 | 33.2 | −26.8 | 8.55847E−09 | 0.000 |
| Normal-27 | 60 | 30.55 | −29.45 | 1.36354E−09 | 0.000 |

TABLE 7

| | |
|---|---|
| Gene | GenBank description |
| HSP105 | AB003334 heat shock 105 kD |

HSP105

| | HSP105 | actin | actin − HSP | 2e(act − HSP) | 2e(act − HSP) × 1000 |
|---|---|---|---|---|---|
| Ca-1 | 31.37 | 24.28 | −7.09 | 0.007340021 | 7.340 |
| Ca-2 | 60 | 34.14 | −25.86 | 1.64197E−08 | 0.000 |
| Ca-3 | 60 | 39.26 | −20.74 | 5.71002E−07 | 0.001 |
| Ca-4 | 60 | 49.1 | −10.9 | 0.000523327 | 0.523 |
| Ca-5 | 27.03 | 20.16 | −6.87 | 0.00854917 | 8.549 |
| Ca-6 | 60 | 34.34 | −25.66 | 1.88612E−08 | 0.000 |
| Ca-7 | 32.63 | 26.07 | −6.56 | 0.010598471 | 10.598 |
| Ca-8 | 60 | 32.56 | −27.44 | 5.49208E−09 | 0.000 |
| Ca-9 | 32.8 | 26.82 | −5.98 | 0.015843117 | 15.843 |
| Ca-10 | 36.85 | 32.1 | −4.75 | 0.037162722 | 37.163 |
| Ca-11 | 36.89 | 32.75 | −4.14 | 0.056719947 | 56.720 |
| Ca-12 | 60 | 35.85 | −24.15 | 5.37187E−08 | 0.000 |
| Ca-13 | 60 | 32.44 | −27.56 | 5.05374E−09 | 0.000 |
| Ca-14 | 30.42 | 32.06 | 1.64 | 3.116658319 | 3116.658 |
| Ca-15 | 35.25 | 28.6 | −6.65 | 0.009957505 | 9.958 |
| Ca-16 | 60 | 33.06 | −26.94 | 7.76698E−09 | 0.000 |
| Ca-17 | 60 | 36 | −24 | 5.96046E−08 | 0.000 |
| Ca-18 | 60 | 39.23 | −20.77 | 5.59251E−07 | 0.001 |
| Ca-19 | 36.63 | 34.6 | −2.03 | 0.244855074 | 244.855 |
| Ca-20 | 60 | 30.84 | −29.16 | 1.66711E−09 | 0.000 |
| Ca-21 | 60 | 28.47 | −31.53 | 3.22496E−10 | 0.000 |
| Ca-22 | 36.34 | 28.14 | −8.2 | 0.003400588 | 3.401 |
| Ca-23 | 34.11 | 27.52 | −6.59 | 0.010380358 | 10.380 |
| Ca-24 | 33.27 | 29.45 | −3.82 | 0.070805243 | 70.805 |
| Ca-25 | 60 | 32.01 | −27.99 | 3.7512E−09 | 0.000 |
| Ca-26 | 30.06 | 22.08 | −7.98 | 0.003960779 | 3.961 |
| Ca-27 | 23.7 | 15.58 | −8.12 | 0.003594483 | 3.594 |
| Ca-28 | 28.39 | 23.07 | −5.32 | 0.025033434 | 25.033 |
| IPMT-1 | 60 | 34.52 | −25.48 | 2.13676E−08 | 0.000 |
| IPMT-2 | 34.01 | 26.76 | −7.25 | 0.006569503 | 6.570 |
| IPMT-3 | 60 | 37.43 | −22.57 | 1.60603E−07 | 0.000 |
| IPMT-4 | 60 | 38.2 | −21.8 | 2.73871E−07 | 0.000 |
| IPMT-5 | 60 | 39.2 | −20.8 | 5.47742E−07 | 0.001 |
| IPMT-6 | 60 | 37.2 | −22.8 | 1.36936E−07 | 0.000 |
| IPMT-7 | 60 | 38.78 | −21.22 | 4.09396E−07 | 0.000 |
| IPMT-8 | 60 | 33.59 | −26.41 | 1.1215E−08 | 0.000 |
| IPMT-9 | 60 | 35.05 | −24.95 | 3.08533E−08 | 0.000 |
| IPMT-10 | 60 | 33.32 | −26.68 | 9.30079E−09 | 0.000 |
| IPMT-11 | 60 | 34.44 | −25.56 | 2.0215E−08 | 0.000 |
| IPMT-12 | 60 | 33.27 | −26.73 | 8.98397E−09 | 0.000 |
| IPMT-13 | 60 | 33.37 | −26.63 | 9.62878E−09 | 0.000 |
| IPMT-14 | 34.81 | 27.29 | −7.52 | 0.005448217 | 5.448 |
| IPMT-15 | 60 | 38.2 | −21.8 | 2.73871E−07 | 0.000 |
| IPMT-16 | 60 | 30.27 | −29.73 | 1.123E−09 | 0.000 |
| IPMT-17 | 36.77 | 30.11 | −6.66 | 0.009888723 | 9.889 |
| Chr. | 60 | 38.52 | −21.48 | 3.41882E−07 | 0.000 |
| Pancreatitis-1 | | | | | |
| Chr. | 60 | 47.5 | −12.5 | 0.000172633 | 0.173 |
| Pancreatitis-2 | | | | | |
| Chr. | 60 | 30.51 | −29.49 | 1.32625E−09 | 0.000 |
| Pancreatitis-3 | | | | | |
| Chr. | 60 | 30.46 | −29.54 | 1.28107E−09 | 0.000 |
| Pancreatitis-4 | | | | | |
| Chr. | 60 | 31.22 | −28.78 | 2.16949E−09 | 0.000 |
| Pancreatitis-5 | | | | | |
| Chr. | 60 | 36.45 | −23.55 | 8.14223E−08 | 0.000 |
| Pancreatitis-6 | | | | | |
| Chr. | 60 | 37.88 | −22.12 | 2.1939E−07 | 0.000 |
| Pancreatitis-7 | | | | | |
| Normal-2 | 60 | 30.53 | −29.47 | 1.34476E−09 | 0.000 |
| Normal-3 | 60 | 36.1 | −23.9 | 6.38827E−08 | 0.000 |
| Normal-4 | 60 | 35.1 | −24.9 | 3.19413E−08 | 0.000 |
| Normal-5 | 60 | 36.46 | −23.54 | 8.19887E−08 | 0.000 |
| Normal-6 | 60 | 34.3 | −25.7 | 1.83455E−08 | 0.000 |
| Normal-7 | 60 | 34.64 | −25.36 | 2.32209E−08 | 0.000 |
| Normal-8 | 60 | 45.34 | −14.66 | 3.86278E−05 | 0.039 |
| Normal-9 | 60 | 41.09 | −18.91 | 2.03013E−06 | 0.002 |
| Normal-10 | 60 | 33.57 | −26.43 | 1.10606E−08 | 0.000 |
| Normal-11 | 60 | 35.7 | −24.3 | 4.8414E−08 | 0.000 |
| Normal-12 | 60 | 33.2 | −26.8 | 8.55847E−09 | 0.000 |
| Normal-13 | 60 | 34.92 | −25.08 | 2.81947E−08 | 0.000 |
| Normal-14 | 60 | 31.42 | −28.58 | 2.49208E−09 | 0.000 |
| Normal-15 | 60 | 35.75 | −24.25 | 5.01213E−08 | 0.000 |
| Normal-16 | 60 | 32.53 | −27.47 | 5.37906E−09 | 0.000 |
| Normal-17 | 60 | 33.08 | −26.92 | 7.8754E−09 | 0.000 |
| Normal-18 | 60 | 40.07 | −19.93 | 1.00109E−06 | 0.001 |
| Normal-19 | 60 | 37.56 | −22.44 | 1.75747E−07 | 0.000 |
| Normal-20 | 60 | 36.2 | −23.8 | 6.84678E−08 | 0.000 |
| Normal-21 | 37.61 | 30.55 | −7.06 | 0.007494251 | 7.494 |
| Normal-22 | 60 | 31.85 | −28.15 | 3.35742E−09 | 0.000 |
| Normal-23 | 60 | 33.52 | −26.48 | 1.06838E−08 | 0.000 |
| Normal-24 | 60 | 37.53 | −22.47 | 1.7213E−07 | 0.000 |

TABLE 8

| | |
|---|---|
| Gene | GenBank description |
| CAPN2 | M23254 calpain, large polypeptide L2 |

CAPN2

| | CAPN2 | actin | actin − CAPN | 2e(act − CAP) | 2e(act − CAP) × 1000 |
|---|---|---|---|---|---|
| Ca-1 | 31.49 | 23.19 | −8.3 | 0.003172861 | 3.173 |
| Ca-2 | 60 | 34.14 | −25.86 | 1.64197E−08 | 0.000 |
| Ca-3 | 60 | 39.26 | −20.74 | 5.71002E−07 | 0.001 |
| Ca-4 | 60 | 49.1 | −10.9 | 0.000523327 | 0.523 |
| Ca-5 | 27.54 | 20.16 | −7.38 | 0.006003419 | 6.003 |
| Ca-6 | 60 | 34.34 | −25.66 | 1.88612E−08 | 0.000 |
| Ca-7 | 32.71 | 24.65 | −8.06 | 0.003747125 | 3.747 |
| Ca-8 | 60 | 32.56 | −27.44 | 5.49208E−09 | 0.000 |
| Ca-9 | 35.15 | 26.82 | −8.33 | 0.003107564 | 3.108 |
| Ca-10 | 39.08 | 30.51 | −8.57 | 0.002631316 | 2.631 |
| Ca-11 | 37.65 | 32.75 | −4.9 | 0.033492921 | 33.493 |
| Ca-12 | 60 | 33.06 | −26.94 | 7.76698E−09 | 0.000 |
| Ca-13 | 60 | 35.85 | −24.15 | 5.37187E−08 | 0.000 |
| Ca-14 | 60 | 32.44 | −27.56 | 5.05374E−09 | 0.000 |
| Ca-15 | 37.87 | 32.06 | −5.81 | 0.017824433 | 17.824 |
| Ca-16 | 37.5 | 27.65 | −9.85 | 0.001083564 | 1.084 |
| Ca-17 | 60 | 36 | −24 | 5.96046E−08 | 0.000 |
| Ca-18 | 60 | 39.23 | −20.77 | 5.59251E−07 | 0.001 |
| Ca-19 | 60 | 40.05 | −19.95 | 9.87306E−07 | 0.001 |
| Ca-20 | 60 | 32.49 | −27.51 | 5.23196E−09 | 0.000 |
| Ca-21 | 39.71 | 27.52 | −12.19 | 0.000214015 | 0.214 |
| Ca-22 | 37.03 | 25.4 | −11.63 | 0.000315516 | 0.316 |
| Ca-23 | 35.42 | 27.43 | −7.99 | 0.00393342 | 3.933 |
| Ca-24 | 38.42 | 29.3 | −9.12 | 0.001797242 | 1.797 |
| Ca-25 | 21.94 | 25.25 | 3.31 | 9.9176616 | 9917.662 |
| Ca-26 | 29.69 | 22.15 | −7.54 | 0.00537321 | 5.373 |
| Ca-27 | 39.22 | 31.85 | −7.37 | 0.006045176 | 6.045 |
| IPMT-1 | 60 | 34.52 | −25.48 | 2.13676E−08 | 0.000 |
| IPMT-2 | 35.41 | 26.76 | −8.65 | 0.002489376 | 2.489 |
| IPMT-3 | 60 | 37.43 | −22.57 | 1.60603E−07 | 0.000 |
| IPMT-4 | 60 | 38.2 | −21.8 | 2.73871E−07 | 0.000 |
| IPMT-5 | 60 | 39.2 | −20.8 | 5.47742E−07 | 0.001 |
| IPMT-6 | 60 | 37.2 | −22.8 | 1.36936E−07 | 0.000 |
| IPMT-7 | 60 | 44.49 | −15.51 | 2.14301E−05 | 0.021 |
| IPMT-8 | 60 | 38.78 | −21.22 | 4.09396E−07 | 0.000 |
| IPMT-9 | 60 | 33.59 | −26.41 | 1.1215E−08 | 0.000 |
| IPMT-10 | 60 | 36.53 | −23.47 | 8.60649E−08 | 0.000 |
| IPMT-11 | 60 | 35.05 | −24.95 | 3.08533E−08 | 0.000 |
| IPMT-12 | 60 | 35.08 | −24.92 | 3.15016E−08 | 0.000 |
| IPMT-13 | 60 | 45.41 | −14.59 | 4.05483E−05 | 0.041 |
| IPMT-14 | 60 | 33.64 | −26.36 | 1.16105E−08 | 0.000 |
| IPMT-15 | 36.26 | 28.59 | −7.67 | 0.004910208 | 4.910 |
| IPMT-16 | 60 | 38.32 | −21.68 | 2.97625E−07 | 0.000 |
| IPMT-17 | 41.24 | 30.27 | −10.97 | 0.000498541 | 0.499 |
| IPMT-18 | 39.15 | 29.54 | −9.61 | 0.001279681 | 1.280 |
| Chr. | 60 | 38.52 | −21.48 | 3.41882E−07 | 0.000 |
| pancreatitis-1 | | | | | |
| Chr. | 60 | 47.5 | −12.5 | 0.000172633 | 0.173 |
| pancreatitis-2 | | | | | |
| Chr. | 60 | 32.1 | −27.9 | 3.99267E−09 | 0.000 |
| pancreatitis-3 | | | | | |
| Chr. | 38.18 | 30.6 | −7.58 | 0.00522628 | 5.226 |
| pancreatitis-4 | | | | | |
| Chr. | 60 | 29.4 | −30.6 | 6.14444E−10 | 0.000 |
| pancreatitis-5 | | | | | |
| Chr. | 60 | 32.2 | −27.8 | 4.27923E−09 | 0.000 |
| pancreatitis-6 | | | | | |
| Chr. | 60 | 37.88 | −22.12 | 2.1939E−07 | 0.000 |
| pancreatitis-7 | | | | | |
| Normal-1 | 60 | 33.39 | −26.61 | 9.76319E−09 | 0.000 |
| Normal-2 | 60 | 35.57 | −24.43 | 4.42423E−08 | 0.000 |
| Normal-3 | 60 | 35.28 | −24.72 | 3.61858E−08 | 0.000 |
| Normal-4 | 60 | 37.95 | −22.05 | 2.30297E−07 | 0.000 |
| Normal-5 | 60 | 39.25 | −20.75 | 5.67058E−07 | 0.001 |
| Normal-6 | 60 | 31.31 | −28.69 | 2.30914E−09 | 0.000 |
| Normal-8 | 60 | 30.4 | −29.6 | 1.22889E−09 | 0.000 |
| Normal-9 | 60 | 34.44 | −25.56 | 2.0215E−08 | 0.000 |
| Normal-10 | 60 | 35.41 | −24.59 | 3.95979E−08 | 0.000 |
| Normal-11 | 60 | 33.21 | −26.79 | 8.618E−09 | 0.000 |
| Normal-12 | 60 | 37.56 | −22.44 | 1.75747E−07 | 0.000 |
| Normal-13 | 60 | 36.2 | −23.8 | 6.84678E−08 | 0.000 |
| Normal-15 | 60 | 30.53 | −29.47 | 1.34476E−09 | 0.000 |
| Normal-16 | 60 | 36.1 | −23.9 | 6.38827E−08 | 0.000 |
| Normal-17 | 60 | 35.1 | −24.9 | 3.19413E−08 | 0.000 |
| Normal-18 | 60 | 36.46 | −23.54 | 8.19887E−08 | 0.000 |
| Normal-19 | 60 | 34.3 | −25.7 | 1.83455E−08 | 0.000 |
| Normal-20 | 60 | 34.64 | −25.36 | 2.32209E−08 | 0.000 |
| Normal-21 | 60 | 45.34 | −14.66 | 3.86278E−05 | 0.039 |
| Normal-22 | 60 | 41.09 | −18.91 | 2.03013E−06 | 0.002 |
| Normal-23 | 60 | 33.57 | −26.43 | 1.10606E−08 | 0.000 |
| Normal-24 | 60 | 35.7 | −24.3 | 4.8414E−08 | 0.000 |
| Normal-25 | 60 | 33.2 | −26.8 | 8.55847E−09 | 0.000 |
| Normal-26 | 60 | 30.55 | −29.45 | 1.36354E−09 | 0.000 |

TABLE 9

| | SOD2 | IGFBP1 | CDKN1C | UBE3A | HSP105 | CAPN2 |
|---|---|---|---|---|---|---|
| Ca | 9/35 | 6/23 | 8/28 | 11/26 | 15/28 | 13/27 |
| IPMT | 4/17 | 0/17 | 0/16 | 1/17 | 3/17 | 3/18 |
| Chr. | 3/8 | 0/7 | 0/6 | 1/7 | 0/7 | 1/7 |
| pancreatitis | | | | | | |
| Normal | 0/27 | 0/23 | 0/25 | 0/25 | 1/23 | 0/24 |

TABLE 10

| | SOD2 | IGFBP1 | CDKN1C | UBE3A | HSP105 | CAPN2 |
|---|---|---|---|---|---|---|
| Ca | 6/35 | 4/23 | 2/28 | 6/26 | 12/28 | 6/27 |
| IPMT | 3/17 | 0/17 | 0/16 | 1/17 | 3/17 | 0/18 |
| Chr. | 0/8 | 0/7 | 0/6 | 0/7 | 0/7 | 1/7 |
| pancreatitis | | | | | | |
| Normal | 0/27 | 0/23 | 0/25 | 0/25 | 1/23 | 0/24 |

TABLE 11

| | | | | | 2e(act − | | | | | | 2e(act − |
|---|---|---|---|---|---|---|---|---|---|---|---|
| | SOD2 | actin | act − SOD | 2e(act − SOD) | SOD) × 1000 | | HSP105 | actin | actin − HSP | 2e(act − HSP) | HSP) × 1000 |
| Ca-1 | 30.43 | 17.54 | −12.89 | 0.00013174 | 0.132 | Ca-1 | 31.37 | 24.28 | −7.09 | 0.00734002 | 7.340 |
| Ca-2 | 27.48 | 14.48 | −13 | 0.00012207 | 0.122 | Ca-2 | 60 | 34.14 | −25.86 | 1.642E−08 | 0.000 |
| Ca-3 | 28.19 | 14.75 | −13.44 | 8.9982E−05 | 0.090 | Ca-3 | 60 | 39.26 | −20.74 | 5.71E−07 | 0.001 |
| Ca-4 | 36.37 | 26.55 | −9.82 | 0.00110633 | 1.106 | Ca-4 | 60 | 49.1 | −10.9 | 0.00052333 | 0.523 |
| Ca-5 | 50 | 33.04 | −16.96 | 7.8439E−06 | 0.008 | Ca-5 | 27.03 | 20.16 | −6.87 | 0.00854917 | 8.549 |
| Ca-6 | 50 | 32.27 | −17.73 | 4.5998E−06 | 0.005 | Ca-6 | 60 | 34.34 | −25.66 | 1.8861E−08 | 0.000 |
| Ca-7 | 50 | 35.89 | −14.11 | 5.6554E−05 | 0.057 | Ca-7 | 32.63 | 26.07 | −6.56 | 0.01059847 | 10.598 |
| Ca-9 | 50 | 34.09 | −15.91 | 1.6241E−05 | 0.016 | Ca-9 | 32.8 | 26.82 | −5.98 | 0.01584312 | 15.843 |
| Ca-10 | 31.94 | 19.27 | −12.67 | 0.00015344 | 0.153 | Ca-10 | 36.86 | 32.1 | −4.75 | 0.03716272 | 37.163 |
| Ca-11 | 50 | 38.8 | −11.2 | 0.00042507 | 0.425 | Ca-11 | 36.89 | 32.75 | −4.14 | 0.05671995 | 56.720 |
| Ca-12 | 50 | 25.44 | −24.56 | 4.043E−08 | 0.000 | Ca-12 | 60 | 35.85 | −24.15 | 5.3719E−08 | 0.000 |
| Ca-13 | 50 | 38.23 | −11.77 | 0.00028634 | 0.286 | Ca-13 | 60 | 32.44 | −27.56 | 5.0537E−09 | 0.000 |
| Ca-14 | 39.54 | 26.36 | −13.18 | 0.00010775 | 0.108 | Ca-14 | 30.42 | 32.06 | 1.64 | 3.11665832 | 3116.658 |
| Ca-16 | 39.33 | 35.35 | −3.98 | 0.06337247 | 63.372 | Ca-16 | 60 | 33.06 | −26.94 | 7.767E−09 | 0.000 |
| Ca-17 | 41.34 | 30.03 | −11.31 | 0.00039387 | 0.394 | Ca-17 | 60 | 36 | −24 | 5.9605E−08 | 0.000 |
| Ca-18 | 39.82 | 34.51 | −5.31 | 0.02520755 | 25.208 | Ca-18 | 60 | 39.23 | −20.77 | 5.5925E−07 | 0.001 |
| Ca-20 | 38.18 | 29.79 | −8.39 | 0.00298098 | 2.981 | Ca-20 | 60 | 30.84 | −29.16 | 1.6671E−09 | 0.000 |
| Ca-21 | 43.07 | 30.19 | −12.88 | 0.00013266 | 0.133 | Ca-21 | 60 | 28.47 | −31.53 | 3.225E−10 | 0.000 |
| Ca-22 | 41.59 | 29.06 | −12.53 | 0.00016908 | 0.169 | Ca-22 | 36.34 | 28.14 | −8.2 | 0.00340059 | 3.401 |
| Ca-24 | 50 | 33.4 | −16.6 | 1.0067E−05 | 0.010 | Ca-24 | 33.27 | 29.45 | −3.82 | 0.07080524 | 70.805 |
| Ca-25 | 50 | 37.78 | −12.22 | 0.00020961 | 0.210 | Ca-25 | 60 | 32.01 | −27.99 | 3.7512E−09 | 0.000 |
| Ca-26 | 42.11 | 37.79 | −4.32 | 0.05006687 | 50.067 | Ca-26 | 30.06 | 22.08 | −7.98 | 0.00396078 | 3.961 |
| Ca-27 | 42.19 | 35.16 | −7.03 | 0.00765172 | 7.652 | Ca-27 | 23.7 | 15.58 | −8.12 | 0.00359448 | 3.594 |
| Ca-28 | 50 | 39.07 | −10.93 | 0.00051256 | 0.513 | Ca-28 | 28.39 | 23.07 | −5.32 | 0.02503348 | 25.033 |
| IPMT-1 | 50 | 35.53 | −14.47 | 4.4065E−05 | 0.044 | IPMT-1 | 60 | 34.52 | −25.48 | 2.1368E−08 | 0.000 |
| IPMT-2 | 50 | 36.05 | −13.95 | 6.3188E−05 | 0.063 | IPMT-2 | 34.01 | 26.76 | −7.25 | 0.0065695 | 6.570 |
| IPMT-4 | 50 | 36.04 | −13.96 | 6.2751E−05 | 0.063 | IPMT-4 | 60 | 38.2 | −21.8 | 2.7387E−07 | 0.000 |
| IPMT-5 | 50 | 34.1 | −15.9 | 1.6354E−05 | 0.016 | IPMT-5 | 60 | 39.2 | −20.8 | 5.4774E−07 | 0.001 |
| IPMT-6 | 50 | 38.11 | −11.89 | 0.00026348 | 0.263 | IPMT-6 | 60 | 37.2 | −22.8 | 1.3694E−07 | 0.000 |
| IPMT-7 | 50 | 35.14 | −14.86 | 3.3627E−05 | 0.034 | IPMT-7 | 60 | 38.78 | −21.22 | 4.094E−07 | 0.000 |
| IPMT-8 | 41.4 | 35.01 | −6.39 | 0.0119239 | 11.924 | IPMT-8 | 60 | 33.59 | −26.41 | 1.1215E−08 | 0.000 |
| IPMT-9 | 50 | 34.07 | −15.93 | 1.6017E−05 | 0.016 | IPMT-9 | 60 | 35.05 | −24.95 | 3.0853E−08 | 0.000 |
| IPMT-11 | 40.8 | 33.77 | −7.03 | 0.00765172 | 7.652 | IPMT-11 | 60 | 34.44 | −25.56 | 2.0215E−08 | 0.000 |
| IPMT-12 | 50 | 35.13 | −14.87 | 3.3395E−05 | 0.033 | IPMT-12 | 60 | 33.27 | −26.73 | 8.984E−09 | 0.000 |
| IPMT-13 | 50 | 33.57 | −16.43 | 1.1326E−05 | 0.011 | IPMT-13 | 60 | 33.37 | −26.63 | 9.6288E−09 | 0.000 |
| IPMT-14 | 40.83 | 31.98 | −8.85 | 0.00216713 | 2.167 | IPMT-14 | 34.81 | 27.29 | −7.52 | 0.00544822 | 5.448 |
| IPMT-15 | 60 | 34.36 | −25.64 | 1.9125E−08 | 0.000 | IPMT-15 | 60 | 38.2 | −21.8 | 2.7387E−07 | 0.000 |
| IPMT-16 | 39.24 | 32.63 | −6.61 | 0.01023745 | 10.237 | IPMT-16 | 60 | 30.27 | −29.73 | 1.123E−09 | 0.000 |
| IPMT-17 | 38.23 | 27.1 | −11.13 | 0.00044621 | 0.446 | IPMT-17 | 36.77 | 30.11 | −6.66 | 0.00988872 | 9.889 |

TABLE 12

| | | | | | 2e(act − | | | | | | |
|---|---|---|---|---|---|---|---|---|---|---|---|
| | | | act − | 2e(act − | SOD) × | | | | actin − | 2e(act − | 2e(act − |
| | SOD2 | actin | SOD | SOD) | 1000 | | HSP105 | actin | HSP | HSP) | HSP) × 1000 |
| chr. pancreatitis-1 | 60 | 35.13 | −24.87 | 3.2612E−08 | 0.000 | Chr. Pancreatitis-1 | 60 | 38.52 | −21.48 | 3.4188E−07 | 0.000 |
| chr. pancreatitis-2 | 60 | 34.39 | −25.61 | 1.9526E−08 | 0.000 | Chr. Pancreatitis-2 | 60 | 47.5 | −12.5 | 0.00017263 | 0.173 |
| chr. pancreatitis-3 | 41.55 | 29.32 | −12.23 | 0.00020816 | 0.208 | Chr. Pancreatitis-3 | 60 | 30.51 | −29.49 | 1.3263E−09 | 0.000 |
| chr. pancreatitis-4 | 43.52 | 34.26 | −9.26 | 0.00163103 | 1.631 | Chr. Pancreatitis-4 | 60 | 30.46 | −29.54 | 1.2811E−09 | 0.000 |
| chr. pancreatitis-5 | 41.13 | 31.67 | −9.46 | 0.0014199 | 1.420 | Chr. Pancreatitis-5 | 60 | 31.22 | −28.78 | 2.1695E−09 | 0.000 |
| chr. pancreatitis-6 | 60 | 33.61 | −26.39 | 1.1372E−08 | 0.000 | Chr. Pancreatitis-6 | 60 | 36.45 | −23.55 | 8.1422E−08 | 0.000 |
| chr. pancreatitis-7 | 41.65 | 33.83 | −7.82 | 0.00442533 | 4.425 | Chr. Pancreatitis-7 | 60 | 37.88 | −22.12 | 2.1939E−07 | 0.000 |
| normal-2 | 60 | 38.34 | −21.66 | 3.0178E−07 | 0.000 | Normal-2 | 60 | 30.53 | −29.47 | 1.3448E−09 | 0.000 |
| normal-3 | 60 | 34.47 | −25.53 | 2.064E−08 | 0.000 | Normal-3 | 60 | 36.1 | −23.9 | 6.3883E−08 | 0.000 |
| normal-4 | 60 | 32.96 | −27.04 | 7.2468E−09 | 0.000 | Normal-4 | 60 | 35.1 | −24.9 | 3.1941E−08 | 0.000 |
| normal-5 | 60 | 32.94 | −27.06 | 7.1471E−09 | 0.000 | Normal-5 | 60 | 36.46 | −23.54 | 8.1989E−08 | 0.000 |
| normal-6 | 60 | 30.57 | −29.43 | 1.3826E−09 | 0.000 | Normal-6 | 60 | 34.3 | −25.7 | 1.8345E−08 | 0.000 |
| normal-7 | 50 | 35.63 | −14.37 | 4.7228E−05 | 0.047 | Normal-7 | 60 | 34.64 | −25.36 | 2.3221E−08 | 0.000 |
| normal-8 | 50 | 39.15 | −10.85 | 0.00054178 | 0.542 | Normal-8 | 60 | 45.34 | −14.66 | 3.8628E−05 | 0.039 |
| normal-9 | 50 | 35.34 | −14.66 | 3.8628E−05 | 0.039 | Normal-9 | 60 | 41.09 | −18.91 | 2.0301E−06 | 0.002 |
| normal-10 | 50 | 36.04 | −13.96 | 6.2751E−05 | 0.063 | Normal-10 | 60 | 33.57 | −26.43 | 1.1061E−08 | 0.000 |
| normal-11 | 50 | 35.81 | −14.19 | 5.3504E−05 | 0.054 | Normal-11 | 60 | 35.7 | −24.3 | 4.8414E−08 | 0.000 |
| normal-12 | 50 | 30.72 | −19.28 | 1.5709E−06 | 0.002 | Normal-12 | 60 | 33.2 | −26.8 | 8.5585E−09 | 0.000 |
| normal-13 | 50 | 32.1 | −17.9 | 4.0885E−06 | 0.004 | Normal-13 | 60 | 34.92 | −25.08 | 2.8195E−08 | 0.000 |
| normal-14 | 50 | 31.05 | −18.95 | 1.9746E−06 | 0.002 | Normal-14 | 60 | 31.42 | −28.58 | 2.4921E−09 | 0.000 |
| normal-15 | 50 | 33.22 | −16.78 | 8.8862E−06 | 0.009 | Normal-15 | 60 | 35.75 | −24.25 | 5.0121E−08 | 0.000 |
| normal-16 | 60 | 34.53 | −25.47 | 2.1516E−08 | 0.000 | Normal-16 | 60 | 32.53 | −27.47 | 5.3791E−09 | 0.000 |
| normal-17 | 60 | 28.2 | −31.8 | 2.6745E−10 | 0.000 | Normal-17 | 60 | 33.08 | −26.92 | 7.8754E−09 | 0.000 |
| normal-19 | 60 | 39.15 | −20.85 | 5.2908E−07 | 0.001 | Normal-19 | 60 | 37.56 | −22.44 | 1.7575E−07 | 0.000 |
| normal-20 | 60 | 38.13 | −21.87 | 2.609E−07 | 0.000 | Normal-20 | 60 | 36.2 | −23.8 | 6.8468E−08 | 0.000 |
| normal-21 | 60 | 34.54 | −25.46 | 2.1666E−08 | 0.000 | Normal-21 | 37.61 | 30.65 | −7.06 | 0.00749426 | 7.494 |
| normal-22 | 60 | 36.06 | −23.94 | 6.2136E−08 | 0.000 | Normal-22 | 60 | 31.85 | −28.15 | 3.3574E−09 | 0.000 |
| normal-23 | 60 | 38.23 | −21.77 | 2.7963E−07 | 0.000 | Normal-23 | 60 | 33.52 | −26.48 | 1.0684E−08 | 0.000 |

TABLE 13

| | | | | | 2e(act − | | | | | | 2e(act − |
|---|---|---|---|---|---|---|---|---|---|---|---|
| | SOD2 | actin | act − SOD | 2e(act − SOD) | SOD) × 1000 | | HSP105 | actin | actin − HSP | 2e(act − HSP) | HSP) × 1000 |
| Ca-1 | 30.43 | 17.54 | −12.89 | 0.000131742 | 0.132 | Ca-1 | 31.37 | 24.28 | −7.09 | 0.007340021 | 7.340 |
| Ca-2 | 27.48 | 14.48 | −13 | 0.00012207 | 0.122 | Ca-2 | 60 | 34.14 | −25.86 | 1.64197E−08 | 0.000 |
| Ca-3 | 28.19 | 14.75 | −13.44 | 8.99823E−05 | 0.090 | Ca-3 | 60 | 39.26 | −20.74 | 5.71002E−07 | 0.001 |
| Ca-4 | 36.37 | 26.55 | −9.82 | 0.001106332 | 1.106 | Ca-4 | 60 | 49.1 | −10.9 | 0.000523327 | 0.523 |
| Ca-5 | 50 | 33.04 | −16.96 | 7.84389E−06 | 0.008 | Ca-5 | 27.03 | 20.16 | −6.87 | 0.00854917 | 8.549 |
| Ca-6 | 50 | 32.27 | −17.73 | 4.59979E−06 | 0.005 | Ca-6 | 60 | 34.34 | −25.66 | 1.88612E−08 | 0.000 |
| Ca-7 | 50 | 35.89 | −14.11 | 5.65544E−05 | 0.057 | Ca-7 | 32.63 | 26.07 | −6.56 | 0.010598471 | 10.598 |
| Ca-9 | 50 | 34.09 | −15.91 | 1.6241E−05 | 0.016 | Ca-9 | 32.8 | 26.82 | −5.98 | 0.015843117 | 15.843 |
| Ca-10 | 31.94 | 19.27 | −12.67 | 0.000153444 | 0.153 | Ca-10 | 36.85 | 32.1 | −4.75 | 0.037162722 | 37.183 |
| Ca-11 | 50 | 38.8 | −11.2 | 0.000425074 | 0.425 | Ca-11 | 36.89 | 32.75 | −4.14 | 0.056719947 | 56.720 |
| Ca-12 | 50 | 25.44 | −24.56 | 4.043E−08 | 0.000 | Ca-12 | 60 | 35.85 | −24.15 | 5.37187E−08 | 0.000 |
| Ca-13 | 50 | 38.23 | −11.77 | 0.000286337 | 0.286 | Ca-13 | 60 | 32.44 | −27.56 | 5.05374E−09 | 0.000 |
| Ca-14 | 39.54 | 26.36 | −13.18 | 0.000107752 | 0.108 | Ca-14 | 30.42 | 32.06 | 1.64 | 3.116658319 | 3116.658 |
| Ca-16 | 39.33 | 35.35 | −3.98 | 0.063372467 | 63.372 | Ca-16 | 60 | 33.06 | −26.94 | 7.76698E−09 | 0.000 |
| Ca-17 | 41.34 | 30.03 | −11.31 | 0.000393868 | 0.394 | Ca-17 | 60 | 36 | −24 | 5.96046E−08 | 0.000 |
| Ca-18 | 39.82 | 34.51 | −5.31 | 0.025207555 | 25.208 | Ca-18 | 60 | 39.23 | −20.77 | 5.59251E−07 | 0.001 |
| Ca-20 | 38.18 | 29.79 | −8.39 | 0.002980975 | 2.981 | Ca-20 | 60 | 30.84 | −29.16 | 1.66711E−09 | 0.000 |
| Ca-21 | 43.07 | 30.19 | −12.88 | 0.000132658 | 0.133 | Ca-21 | 60 | 28.47 | −31.53 | 3.22496E−10 | 0.000 |
| Ca-22 | 41.59 | 29.06 | −12.53 | 0.000169081 | 0.169 | Ca-22 | 36.34 | 28.14 | −8.2 | 0.003400588 | 3.401 |
| Ca-24 | 50 | 33.4 | −16.6 | 1.0067E−05 | 0.010 | Ca-24 | 33.27 | 29.45 | −3.82 | 0.070805243 | 70.805 |
| Ca-25 | 50 | 37.78 | −12.22 | 0.000209611 | 0.210 | Ca-25 | 60 | 32.01 | −27.99 | 3.7512E−09 | 0.000 |
| Ca-26 | 42.11 | 37.79 | −4.32 | 0.050066857 | 50.067 | Ca-26 | 30.06 | 22.08 | −7.98 | 0.003960779 | 3.961 |
| Ca-27 | 42.19 | 35.16 | −7.03 | 0.007651721 | 7.652 | Ca-27 | 23.7 | 15.58 | −8.12 | 0.003594483 | 3.594 |
| Ca-28 | 50 | 39.07 | −10.93 | 0.000512557 | 0.513 | Ca-28 | 28.39 | 23.07 | −5.32 | 0.025033434 | 25.033 |
| IPMT-1 | 50 | 35.53 | −14.47 | 4.40652E−05 | 0.044 | IPMT-1 | 60 | 34.52 | −25.48 | 2.13676E−08 | 0.000 |
| IPMT-2 | 50 | 36.05 | −13.95 | 6.31876E−05 | 0.063 | IPMT-2 | 34.01 | 26.76 | −7.25 | 0.006569503 | 6.570 |
| IPMT-4 | 50 | 36.04 | −13.96 | 6.27511E−05 | 0.063 | IPMT-4 | 60 | 38.2 | −21.8 | 2.73871E−07 | 0.000 |
| IPMT-5 | 50 | 34.1 | −15.9 | 1.6354E−05 | 0.016 | IPMT-5 | 60 | 39.2 | −20.8 | 5.47742E−07 | 0.001 |
| IPMT-6 | 50 | 38.11 | −11.89 | 0.000263483 | 0.263 | IPMT-6 | 60 | 37.2 | −22.8 | 1.36936E−07 | 0.000 |
| IPMT-7 | 50 | 35.14 | −14.86 | 3.36275E−05 | 0.034 | IPMT-7 | 60 | 38.78 | −21.22 | 4.09396E−07 | 0.000 |
| IPMT-8 | 41.4 | 35.01 | −6.39 | 0.0119239 | 11.924 | IPMT-8 | 60 | 33.59 | −26.41 | 1.1215E−08 | 0.000 |
| IPMT-9 | 50 | 34.07 | −15.93 | 1.60174E−05 | 0.016 | IPMT-9 | 60 | 35.05 | −24.95 | 3.08533E−08 | 0.000 |
| IPMT-11 | 40.8 | 33.77 | −7.03 | 0.007651721 | 7.652 | IPMT-11 | 60 | 34.44 | −25.56 | 2.0215E−08 | 0.000 |
| IPMT-12 | 50 | 35.13 | −14.87 | 3.33952E−05 | 0.033 | IPMT-12 | 60 | 33.27 | −26.73 | 8.98397E−09 | 0.000 |
| IPMT-13 | 50 | 33.57 | −16.43 | 1.1326E−05 | 0.011 | IPMT-13 | 60 | 33.37 | −26.63 | 9.62878E−09 | 0.000 |
| IPMT-14 | 40.83 | 31.98 | −8.85 | 0.002167128 | 2.167 | IPMT-14 | 34.81 | 27.29 | −7.52 | 0.005448217 | 5.448 |
| IPMT-15 | 60 | 34.36 | −25.64 | 1.91245E−08 | 0.000 | IPMT-15 | 60 | 38.2 | −21.8 | 2.73871E−07 | 0.000 |
| IPMT-16 | 39.24 | 32.63 | −6.61 | 0.010237448 | 10.237 | IPMT-16 | 60 | 30.27 | −29.73 | 1.123E−09 | 0.000 |
| IPMT-17 | 38.23 | 27.1 | −11.13 | 0.000446207 | 0.446 | IPMT-17 | 36.77 | 30.11 | −6.66 | 0.009888723 | 9.889 |

TABLE 14

| | | | | | 2e(act − | | | | | | |
|---|---|---|---|---|---|---|---|---|---|---|---|
| | | | act − | 2e(act − | SOD) × | | | | actin − | 2e(act − | 2e(act − |
| | SOD2 | actin | SOD | SOD) | 1000 | | HSP105 | actin | HSP | HSP) | HSP) × 1000 |
| chr. pancreatitis-1 | 60 | 35.13 | −24.87 | 3.26125E−08 | 0.000 | Chr. Pancreatitis-1 | 60 | 38.52 | −21.48 | 3.41882E−07 | 0.000 |
| chr. pancreatitis-2 | 60 | 34.39 | −25.61 | 1.95264E−08 | 0.000 | Chr. Pancreatitis-2 | 60 | 47.5 | −12.5 | 0.000172633 | 0.173 |
| chr. pancreatitis-3 | 41.55 | 29.32 | −12.23 | 0.000208163 | 0.208 | Chr. Pancreatitis-3 | 60 | 30.51 | −29.49 | 1.32625E−09 | 0.000 |
| chr. pancreatitis-4 | 43.52 | 34.26 | −9.26 | 0.001631031 | 1.631 | Chr. Pancreatitis-4 | 60 | 30.46 | −29.54 | 1.28107E−09 | 0.000 |
| chr. pancreatitis-5 | 41.13 | 31.67 | −9.46 | 0.001419895 | 1.420 | Chr. Pancreatitis-5 | 60 | 31.22 | −28.78 | 2.16949E−09 | 0.000 |
| chr. pancreatitis-6 | 60 | 33.61 | −26.39 | 1.13715E−08 | 0.000 | Chr. Pancreatitis-6 | 60 | 36.45 | −23.55 | 8.14223E−08 | 0.000 |
| chr. pancreatitis-7 | 41.65 | 33.83 | −7.82 | 0.004425328 | 4.425 | Chr. Pancreatitis-7 | 60 | 37.88 | −22.12 | 2.1939E−07 | 0.000 |
| normal-2 | 60 | 38.34 | −21.66 | 3.0178E−07 | 0.000 | Normal-2 | 60 | 30.53 | −29.47 | 1.34476E−09 | 0.000 |
| normal-3 | 60 | 34.47 | −25.53 | 2.06397E−08 | 0.000 | Normal-3 | 60 | 36.1 | −23.9 | 6.38827E−08 | 0.000 |
| normal-4 | 60 | 32.96 | −27.04 | 7.24684E−09 | 0.000 | Normal-4 | 60 | 35.1 | −24.9 | 3.19413E−08 | 0.000 |
| normal-5 | 60 | 32.94 | −27.06 | 7.14707E−09 | 0.000 | Normal-5 | 60 | 36.46 | −23.54 | 8.19887E−08 | 0.000 |
| normal-6 | 60 | 30.57 | −29.43 | 1.38257E−09 | 0.000 | Normal-6 | 60 | 34.3 | −25.7 | 1.83455E−08 | 0.000 |
| normal-7 | 50 | 35.63 | −14.37 | 4.72279E−05 | 0.047 | Normal-7 | 60 | 34.64 | −25.36 | 2.32209E−08 | 0.000 |
| normal-8 | 50 | 39.15 | −10.85 | 0.000541782 | 0.542 | Normal-8 | 60 | 45.34 | −14.66 | 3.86278E−05 | 0.039 |
| normal-9 | 50 | 35.34 | −14.66 | 3.86278E−05 | 0.039 | Normal-9 | 60 | 41.09 | −18.91 | 2.03013E−06 | 0.002 |
| normal-10 | 50 | 36.04 | −13.96 | 6.27511E−05 | 0.063 | Normal-10 | 60 | 33.57 | −26.43 | 1.10606E−08 | 0.000 |
| normal-11 | 50 | 35.81 | −14.19 | 5.35038E−05 | 0.054 | Normal-11 | 60 | 35.7 | −24.3 | 4.8414E−08 | 0.000 |
| normal-12 | 50 | 30.72 | −19.28 | 1.57088E−06 | 0.002 | Normal-12 | 60 | 33.2 | −26.8 | 8.55847E−09 | 0.000 |
| normal-13 | 50 | 32.1 | −17.9 | 4.08849E−06 | 0.004 | Normal-13 | 60 | 34.92 | −25.08 | 2.81947E−08 | 0.000 |
| normal-14 | 50 | 31.05 | −18.95 | 1.97461E−06 | 0.002 | Normal-14 | 60 | 31.42 | −28.58 | 2.49208E−09 | 0.000 |
| normal-15 | 50 | 33.22 | −16.78 | 8.88621E−06 | 0.009 | Normal-15 | 60 | 35.75 | −24.25 | 5.01213E−08 | 0.000 |
| normal-16 | 60 | 34.53 | −25.47 | 2.15162E−08 | 0.000 | Normal-16 | 60 | 32.53 | −27.47 | 5.37906E−09 | 0.000 |
| normal-17 | 60 | 28.2 | −31.8 | 2.67452E−10 | 0.000 | Normal-17 | 60 | 33.08 | −26.92 | 7.8754E−09 | 0.000 |
| normal-19 | 60 | 39.15 | −20.85 | 5.29084E−07 | 0.001 | Normal-19 | 60 | 37.56 | −22.44 | 1.75747E−07 | 0.000 |
| normal-20 | 60 | 38.13 | −21.87 | 2.609E−07 | 0.000 | Normal-20 | 60 | 36.2 | −23.8 | 6.84678E−08 | 0.000 |
| normal-21 | 60 | 34.54 | −25.46 | 2.16659E−08 | 0.000 | Normal-21 | 37.61 | 30.55 | −7.06 | 0.007494251 | 7.494 |
| normal-22 | 60 | 36.06 | −23.94 | 6.21358E−08 | 0.000 | Normal-22 | 60 | 31.85 | −28.15 | 3.35742E−09 | 0.000 |
| normal-23 | 60 | 38.23 | −21.77 | 2.79626E−07 | 0.000 | Normal-23 | 60 | 33.52 | −26.48 | 1.06838E−08 | 0.000 |

### INDUSTRIAL APPLICABILITY

We have demonstrated that a mere comparison between normal and cancerous tissues of pancreas is not a good approach for the analyses of transformation process. In contrast, through the screening with the fractionated ductal cells of normal and carcinoma-origin, we could identify a set of genes that may be useful in the diagnosis of PDC.

Among the thirty-eight genes identified, a few of them were already known to be highly expressed in carcinoma cells. PTPRU was, for instance, identified through the effort to isolate novel protein-tyrosine phosphatases from pancreatic carcinoma cell lines (Wang, H., Lian, Z., Lerch, M. M., Chen, Z., Xie, W., and Ullrich, A. Characterization of PCP-2, a novel receptor protein tyrosine phosphatase of the MAM domain family. Oncogene, 12: 2555-2562, 1996.). Although Wang et al. demonstrated the presence of its expression both in normal pancreas tissue and pancreas carcinoma cell lines, our current study has restricted the expression of PTPRU to the ductal cells from cancer patients. The discrepancy among these observations may be due to the difference in the assay system; comparison of whole tissues or fractionated ductal cells.

CEACAM7 belongs to the CEA family of proteins. In contrast to the high expression of CEA in the colorectal carcinomas, CEACAM7 was shown to be abundantly expressed in normal colon epithelium, but its expression was reported to be down-regulated upon malignant transformation (Scholzel, S., Zimmermann, W., Schwarzkopf, G., Grunert, F., Rogaczewski, B., and Thompson, J. Carcinoembryonic antigen family members CEACAM6 and CEACAM7 are differentially expressed in normal tissues and oppositely deregulated in hyperplastic colorectal polyps and early adenomas. Am. J. Pathol., 156: 595-605, 2000.; Thompson, J., Seitz, M., Chastre, E., Ditter, M., Aldrian, C., Gespach, C., and Zimmermann, W. Down-regulation of carcinoembryonic antigen family member 2 expression is an early event in colorectal tumorigenesis. Cancer Res., 57: 1776-1784, 1997.). Although its expression in pancreas has not been documented well, CEACAM7 protein may be found within the normal pancreatic ductal cells (Scholzel, S., Zimmermann, W., Schwarzkopf, G., Grunert, F., Rogaczewski, B., and Thompson, J. Carcinoembryonic antigen family members CEACAM6 and CEACAM7 are differentially expressed in normal tissues and oppositely deregulated in hyperplastic colorectal polyps and early adenomas. Am. J. Pathol., 156: 595-605, 2000.). However, our observation for the cancer-specific expression of CEACAM7 may open a possibility of this gene as a novel cancer marker both in the serum and the ductal cell-based assays.

AC133 was initially identified as a cell surface marker specific to hematopoietic stem cell-enriched fraction that exhibits CD34^{high}, CD38^{low/nog} and c-kit⁺ phenotype (Hin, A. H., Miraglia, S., Zanjani, E. D., Almeida-Porada, G., Ogawa, M., Leary, A. G., Olweus, J., Kearney, J., and Buck, D. W. AC133, a novel marker for human hematopoietic stem and progenitor cells. Blood, 90: 5002-5012, 1997.). AC133 is also expressed on the precursor of endothelial cells (Gallacher, L., Murdoch, B., Wu, D. M., Karanu, F. N., Keeney, M., and Bhatia, M. Isolation and characterization of human CD34(−)Lin(−) and CD34(+)Lin(−) hematopoietic stem cells using cell surface markers AC133 and CD7. Blood, 95: 2813-2820, 2000.), indicating that AC133 may be a marker for very immature hemangioblast, a common precursor for blood cells and blood vessels. Expression of AC133 in the tissues other than bone marrow and retina has not been documented, and our study would be the first one to identify AC133 expression in the pancreatic ductal cell-lineage. Given the abundant expression of AC133 in the normal, not transformed, hemangioblasts, its expression in the cancer ductal cells may imply that AC133 is also a marker to the precursor for ductal cells. Increase of AC133 expression in PDC may reflect the immature nature of cancer cells in the differentiation program of ductal cells.

M1S1 or gastrointestinal tumor-associated antigen 1 (GA733-1) was originally identified as a tumor-associated antigen on a stomach adenocarcinoma cell line, and was shown to be also expressed in pancreatic carcinoma cell lines (Linnenbach, A. J., Wojcierowski, J., Wu, S., Pyrc, J. J., Ross, A. H., Dietzschold, B., Speicher, D., and Koprowski, H. Sequence investigation of the major gastrointestinal tumor-associated antigen gene family, GA733. Proc. Natl. Acad. Sci. USA, 86: 27-31, 1989′.). MMP9 catalyzes the degradation of extracellular matrix, and its expression may contribute to the mobilization of hematopoietic stem cells (Pruijt, J. F., Fibbe, W. E., Laterveer, L., Pieters, R. A., Lindley, I. J., Paemen, L., Masure, S., Willemze, R., and Opdenakker, G. Prevention of interleukin-8-induced mobilization of hematopoietic progenitor cells in rhesus monkeys by inhibitory antibodies against the metalloproteinase gelatinase B (MMP-9). Proc. Natl. Acad. Sci. USA, 96: 10863-10868, 1999.) and to the invasive property of carcinoma cells (Turner, H. E., Nagy, Z., Esiri, M. M., Harris, A. L., and Wass, J. A. Role of matrix metalloproteinase 9 in pituitary tumor behavior. J. Clin. Endocrinol. Metab., 85: 2931-2935, 2000.).

In conclusion, DNA microarray analysis with purified ductal cell fractions has been proved to be an efficient and superior approach to extract the PDC-specific genes, when compared to a mere comparison of tissue specimens. Our current data have paved a way to the ERCP-based sensitive and specific test for the detection of pancreatic cancer.

What is claimed is:

1. A method for identifying a pancreatic ductal carcinoma-specific gene, said method comprising the steps of:
(a) preparing pancreatic ductal cells from a pancreatic ductal carcinoma patient and a normal individual;
(b) detecting the gene expression in the pancreatic ductal cells prepared from the pancreatic ductal carcinoma patient and the gene expression in the pancreatic ductal cells prepared from the normal individual;
(c) comparing the gene expression in the pancreatic ductal cells prepared from the pancreatic ductal carcinoma patient with the gene expression in the pancreatic ductal cells prepared from the normal individual; and
(d) identifying a gene specifically expressed in the pancreatic ductal carcinoma patient and a gene specifically expressed in the normal individual.

2. The method of claim 1, wherein the pancreatic ductal cells are prepared from a pancreatic juice.

3. The method of claim 1, wherein the pancreatic ductal cells are prepared using MUC1 gene expression as an index.

4. A method of testing for pancreatic ductal carcinoma, said method comprising the steps of:
(a) preparing a tissue or cells from a subject;
(b) detecting, in the tissue or cells, expression of a pancreatic ductal carcinoma-specific gene identified by the method of claim 1; and
(c) comparing the expression detected in step (b) with expression of the gene in a control tissue or control cells,
wherein the subject is suspected of having pancreatic ductal carcinoma if the expression detected in step (b) is significantly higher than the expression of the gene in the control tissue or control cells, where the gene is specifically expressed in a pancreatic ductal carcinoma patient, or if the expression detected in step (b) is significantly lower than the expression of the gene in the control tissue or control cells, where the gene is specifically expressed in a normal individual.

5. The method of claim 4, wherein the pancreatic ductal carcinoma-specific gene is the CEACAM7 gene, the AC133 gene, the SOD2 gene, the CDKN1C gene, the HSP105 gene, the IGFBP1 gene, the UBE3A gene, or the CAPN2 gene, or a combination of two or more of the genes.

6. The method of claim 4 or 5, wherein the cells prepared from the subject are pancreatic ductal cells.

7. The method of claim 6, wherein the pancreatic ductal cells are prepared from a pancreatic juice.

8. The method of claim 6, wherein the pancreatic ductal cells are prepared using MUC1 gene expression as an index.

9. A drug for testing for pancreatic ductal carcinoma, said drug comprising, as an active ingredient, a molecule selected from the group consisting of:
(a) an antibody binding to a protein encoded by a pancreatic ductal carcinoma-specific gene identified by the method of claim 1; and
(b) an oligonucleotide specifically hybridizing to a transcription product of a pancreatic ductal carcinoma-specific gene identified by the method of claim 1.

10. The drug of claim 9, wherein the pancreatic ductal carcinoma-specific gene is the CEACAM7 gene, the AC133 gene, the SOD2 gene, the CDKN1C gene, the HSP105 gene, the IGFBP1 gene, the UBE3A gene, or the CAPN2 gene, or a combination of two or more of the genes.

11. A method of testing for pancreatic ductal carcinoma, said method comprising the step of:
(a) detecting, in a subject, a genetic polymorphism or mutation that causes abnormal expression of a pancreatic ductal carcinoma-specific gene identified by the method of claim 1 or abnormal activity of a protein encoded by the gene;
wherein the subject is suspected of having pancreatic ductal carcinoma if the subject has the genetic polymorphism or mutation.

12. The method of claim 11, wherein the pancreatic ductal carcinoma-specific gene is the CEACAM7 gene, the AC133 gene, the SOD2 genes the CDKNLC gene, the HSP105 gene, the IGFBP1 gene, the UBE3A gene, or the CAPN2 gene, or a combination of two or more of the genes.

13. A method for identifying a drug candidate compound for treating or preventing pancreatic ductal carcinoma, said method comprising the steps of:
(a) administering a test compound to a test animal or test cells or contacting the test compound with the test animal or test cells; and
(b) detecting, in the test animal or test cells, expression of a pancreatic ductal carcinoma-specific gene identified by the method of claim 1;
wherein the test compound is judged to be a drug candidate compound for treating or preventing pancreatic ductal carcinoma if the test compound decreases the expression detected in step (b), where the gene is specifically expressed in a pancreatic ductal carcinoma patient, or if the test compound increases the expression detected in step (b), where the gene is specifically expressed in a normal individual.

14. A method for identifying a drug candidate compound for treating or preventing pancreatic ductal carcinoma, said method comprising the steps of:
(a) administering a test compound to a test animal or test cells harboring a reporter gene operably linked to the expression control region of a pancreatic ductal carcinoma-specific gene identified by the method of claim 1 or contacting the test compound with the test animal or test cells; and
(b) detecting, in the test animal or test cells, expression of the reporter gene;
wherein the test compound is judged to be a drug candidate compound for treating or preventing pancreatic ductal carcinoma if the test compound decreases the expression detected in step (b), where the pancreatic ductal carcinoma-specific gene is specifically expressed in a pancreatic ductal carcinoma patient, or if the test compound increases the expression detected in step (b), where the pancreatic ductal carcinoma-specific gene is specifically expressed in a normal individual.

15. A method for identifying a drug candidate compound for treating or preventing pancreatic ductal carcinoma, said method comprising the steps of:
(a) contacting a test compound with a protein encoded by a pancreatic ductal carcinoma-specific gene identified by the method of claim 1; and
(b) detecting activity of the protein;
wherein the test compound is judged to be a drug candidate compound for treating or preventing pancreatic ductal carcinoma if the test compound decreases the activity detected in step (b), where the gene is specifically expressed in a pancreatic ductal carcinoma patient, or if the test compound increases the activity detected in step (b), where the gene is specifically expressed in a normal individual.

16. The method of any one of claims 13 to 15, wherein the pancreatic ductal carcinoma-specific gene is the CEACAM7 gene, the AC133 gene, the SOD2 gene, the CDKN1C gene, the HSP105 gene, the IGFBP1 gene, the UBE3A gene, or the CAPN2 gene, or a combination of two or more of the genes.

17. A drug for treating or preventing pancreatic ductal carcinoma, said drug comprising, as an active ingredient, a compound identified by the method of any one of claims 13 to 15.
